SEQUENCE LISTING (1 sequence)

SEQ ID NO: 1 - protein, 16 aa, Artificial
  Synthetic sequence of BH3 domain peptide from Bak
GQVGRQLAIIGDDINR

(19) United States
(12) Patent Application Publication
(10) Pub. No.: US 2010/0113466 A1
(43) Pub. Date: May 6, 2010

(54) OXAZOLE-PYRIDAZINE-OXAZOLE ALPHA-HELIX MIMETIC

(75) Inventors: Julius Rebek, JR., La Jolla, CA (US); Lionel Moisan, St. Quentin en Yvelines Cedex (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Scripps Research Institute; Office of Patent Counsel, La Jolla, CA (US)

(21) Appl. No.: 12/575,952

(22) Filed: Oct. 8, 2009

Related U.S. Application Data
(60) Provisional application No. 61/195,625, filed on Oct. 8, 2008.

(51) Int. Cl. A61K 31/501; C07D 403/14; A61P 35/00

(52) U.S. Cl. 514/252.03; 544/238; 514/252.05

(57) ABSTRACT

There are provided alpha helix scaffolds mimicking i, i+3/i+4, i+7 or i+11 residues having the general structure oxazole-pyridazine-piperidine or oxazole-pyridazine-oxazole. The common pyridazine heterocycle originates from substituted or unsubstituted dimethyl 1,2,4,5-tetrazine-3,6-dicarboxylate. These scaffolds are synthetic counterparts of amphiphilic alpha helices having a hydrophilic face along one side and a hydrophobic face along the other side of the helix.

### CROSS-REFERENCES TO RELATED APPLICATIONS

This application claims the benefit of U.S. Provisional Application No. 61/195,625, filed Oct. 8, 2008, which is incorporated herein by reference in its entirety and for all purposes.

### BACKGROUND OF THE INVENTION

By the present application there are provided nonpeptidic scaffolds that serve as alpha-helix mimetics. More particularly, there are provided compounds, intermediates and methods for the preparation and uses thereof, and pharmaceutical compositions comprising nonpeptidic alpha-helix mimetics having an oxazole-pyridazine-piperidine scaffold or an oxazole-pyridazine-oxazole scaffold.

Protein-protein interactions are involved in the regulation of a wide variety of biological processes. Since the sequencing of the human genome, some research groups have put forward the challenge of developing a small molecule inhibitor for every protein-protein interaction. See Schreiber, Bioorg. Med. Chem. 1998, 6:1127. Without wishing to be bound by any theory, this is considered to be unlikely for many protein-protein complexes, due, for example, to the large surface areas involved [e.g., ˜1600 Å (Jones, et al., Proc. Natl. Acad. Sci. U.S.A., 1996, 93:13), the number of atoms involved [e.g., ≧170 atoms (Bogan, et al., J. Mol. Biol., 1998, 280:1)] and/or the relatively flat shapes of the proteins involved. Two exceptions are possible. Allosteric sites, particularly those deep within the core of a protein, can modify protein-protein interactions such as those well known in the association of hemoglobin subunits (Dickerson, et al., In: Hemoglobin: Structure, Function, Evolution, and Pathology; Benjamin Cummings, 1983; pp 48-58). Additionally, another favorable situation arises when one of the interacting surfaces features a deep, narrow invagination. An appropriate small molecule can be essentially surrounded in such an environment, with the consequence of high binding affinity through the molecular recognition elements of the small molecule and the protein(s). The natural complement for the cleft may be a strand or loop structure, (Davis, et al., Proc. Natl. Acad. Sci. U.S.A., 2006, 103:2953) but alpha-helices are often involved (Tsai, et al., Protein Sci., 1997, 6:1793). Moreover, only a few side chains of the helices typically occupy the binding site on complexation.

As well known in the art, alpha helices present the side chains of the residues thereof along a rod-like helical structure. Approximately 3.6 amino acid residues make up a single turn of an alpha-helix. Thus, side chains that are adjacent in space form a “side” of an alpha-helix with residues which occur every three to four residues along the linear amino acid sequence. As customary in the art, this spacing can be referred to as “i, i+3/i+4, i+7” and the like to indicate that the side chains of residues offset from residue “i” lie approximately along a side of the alpha helix, in spatial proximity. The term “face” in the context of alpha helices is synonomous with the term “side.” Without wishing to be bound by any theory, it is believed that the i, i+3/i+4 and i+7 residues can make crucial contacts with a target protein, and that such contacts constitute the majority of binding energy (Fairlie, et al., Curr. Med. Chem., 1998, 5:29; Sattler, et al., Science, 1997, 275:983). As known in the art, the alpha-helix conformation is stabilized by steric interactions along the backbone as well as hydrogen bonding interactions between the backbone amide carbonyls and NH groups of each amino acid. The side chains of an alpha helix project with well known distances and angular relationships. See Fairlie, et al., Curr. Med. Chem., 1998, 5:29-62; Jain et al., Mol. Divers., 2004, 8:89-100; Cochran, Curr. Opin. Chem. Biol., 2001, 5:654-659; Zutshi, et al., Curr. Opin. Chem. Biol., 1998, 2:62-66; Toogood, J. Med. Chem., 2002, 5:1543-1558; Berg, Angew. Chem. Int. Ed., 2003, 42:2462-2481.

The syntheses of peptidomimetics having a stabilized alpha-helical conformation have been achieved by introducing synthetic templates into the peptidic chain (Kemp, et al., J. Am. Chem. Soc., 1996, 118:4240-4248; Austin, et al., J. Am. Chem. Soc., 1997, 119:6461-6472), by using β-hairpin mimetics (Fasan, et al., Angew. Chem. Int. Ed., 2004, 43:2109-2112), β-peptide sequences (Kritzer, et al., J. Am. Chem. Soc., 2004, 126:9468-9469), and unnatural oligomers with discrete folding propensities (foldamers) (Sadowsky, et al., J. Am. Chem. Soc., 2005, 127:11966-11968). Small synthetic molecules able to mimic the surfaces of constrained peptides offer the advantage of improved stability, lower molecular weight and in some cases better bioavailability. Synthetic small molecules that adopt various well-defined secondary structures are well-documented. See e.g., Hagihara, et al. J. Am. Chem. Soc., 1992, 114:6568-6570; Gennari, et al. Angew. Chem. Int. Ed. Engl., 1994, 33:2067-2069; Gude, et al. Tetrahedron Lett., 1996, 37:8589-8592; Cho, et al., Science, 1993, 261:1303-1305; Hamuro, et al., J. Am. Chem. Soc., 1996, 118:7529-7541; Nowick, et al., J. Am. Chem. Soc., 1996, 118:1066-1072; Lokey & Iverson, Nature, 1995, 375:303-305; Murray & Zimmerman, J. Am. Chem. Soc., 1992, 114:4010-4011; Antuch, et al., Bioorg. Med. Chem. Lett., 2006, 16:1740-1743. For reviews concerning alpha-helix mimetics, see e.g., Yin & Hamilton, Angew. Chem. Int. Ed., 2005, 44:4130-4163; Fletcher & Hamilton, J. R. Soc. Interface, 2006, 3:215-233; Davis, et al. Chem. Soc. Rev., 2007, 36:326-334. See also Cummins, et al., Chem. Biol. Drug Des., 2006, 67:201-205; Ahn & Han, Tetrahedron Lett., 2007, 48:3543-3547.

The development of non-peptide based scaffolds capable of displaying functionality in a fashion imitating the relevant binding residues of an alpha-helix was pioneered by Hamilton and co-workers. See e.g., Davis, et al., Chem. Soc. Rev. 2007, 36:326; Fletcher, et al., J. R. Soc. Interface 2006, 3:215; Yin, et al., Angew. Chem. Int. Ed. 2005, 44:4130; Antuch, et al., Bioorg. Med. Chem. Lett. 2006, 16:1740; Arkin, et al., Nat. Rev. Drug Disc. 2004, 3:301; Babine, et al., Chem. Rev. 1997, 97:1359; and Walensky, Cell Death Differ. 2006, 1. Indeed, the first useful mimetics for an alpha-helix were reported only recently by Hamilton and coworkers. These include the terphenyl scaffold (Orner, et al., J. Am. Chem. Soc., 2001, 123:5382-5383; Yin, et al., J. Am. Chem. Soc., 2005, 127:10191-10196; Yin, et al., Angew. Chem. Int. Ed., 2005, 44:2704-2707), and its pyridine (Ernst, et al., Angew. Chem. Int. Ed., 2003, 42:535-539) and terephthalic acid (Yin & Hamilton, Bioorg. Med. Chem. Lett., 2004, 14:1375-1379) analogues.

Many of these compounds have shown relatively high affinity for alpha-helix binding sites, as well as in vitro and in vivo activity. For example, terphenyl derivatives functionalized at the 3, 2′, and 2″ positions such as HA1 (below) can achieve a staggered conformation wherein the substituents are displayed in a way that closely resembles the spatial positioning of i, i+3, and i+7 residues of an alpha-helix. A series of these molecules was synthesized in a modular fashion by Hamilton and coworkers, allowing for the incorporation of multiple components. See Yin, et al., J. Am. Chem. Soc., 2005, Id.

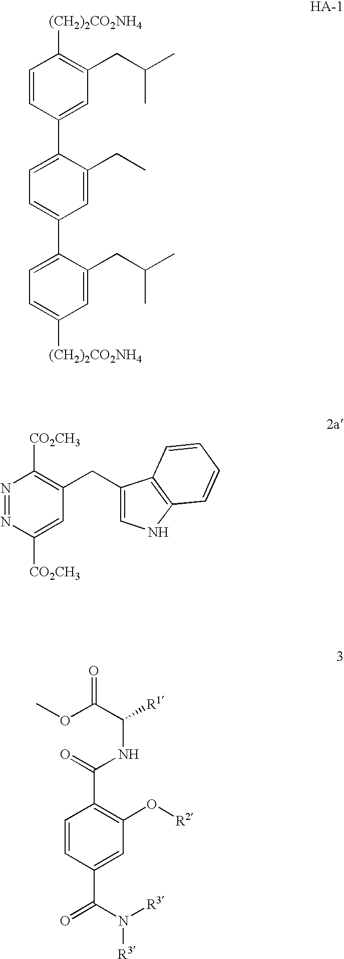

In connection with studies on the synthesis of heterocyclic alpha-helix mimetics (Biros, et al., Bioorg. Med. Chem. Lett., 2007, 17:4641-4645; Volonterio, et al., Org. Lett., 2007, 9:3733-3736; Moisan, et al., Heterocycles, 2007, 73:661-671), the preparation of the 3,4,6-trisubstituted pyridazine 2a′ (above), bearing an indole side chain was required. This structure is inspired by Hamilton's terephthalamide scaffold 3 (above), which is known to disrupt protein-protein interactions (Yin & Hamilton, Bioorg. Med. Chem. Lett., 2004, 14:1375-1379; Yin, et al., J. Am. Chem. Soc., 2005, 127:5463-5468) when R^{1′}, R^{2′} and R^{3′} are typically side chains of hydrophobic amino acids. The pyridazine scaffold offers remote hydrophilic sites, regioselective functionalization (Biros, et al., Bioorg. Med. Chem. Lett., 2007, 17:4641-4645; Volonterio, et al., Org. Lett., 2007, 9:3733-3736; Moisan, et al., Heterocycles, 2007, 73:661-671) and a variety of amino acid side chains for small library synthesis. Further pyridazine based alpha-helix mimetics having a variety of functional groups are disclosed in U.S. Provisional Patent application Ser. No. 60/965,100, filed Aug. 18, 2007, incorporated herein by reference in its entirety and for all purposes.

In addition to the above pyridazine based alpha-helix mimetics, the pyridazine ring is also encountered as a structural component of other compounds possessing a variety of biological activities including analgesic (Rohet, et al., Bioorg. Med. Chem., 1997, 5:655-659), antibacterial (Tucker, et al., J. Med. Chem., 1998, 41:3727-3735), antiinflammatory (Tamayo, et al., Bioorg. Med. Chem. Lett., 2005, 15:2409-2413), antihypertensive (Benson, et al., J. Org. Chem., 1987, 52:4610-4614) and antihistaminic (Gyoten, et al., Chem. Pharm. Bull., 2003, 51:122-133). This heterocycle is also useful for the preparation of other heterocycles (Naud, et al., Eur. J. Org. Chem., 2007, 3296-3310), π-conjugated organic materials with desirable electronic properties (Yasuda, et al., Chem. Mater., 2005, 17:6060-6068) and self-assembled supramolecular architectures (Cuccia, et al., Angew. Chem. Int. Ed., 2000, 39:233-237). These pharmacological and technological properties of pyridazines encourage the development of methods for their synthesis and functionalization (Nara, et al., Synlett, 2006, 3185-3204). In particular, the Inverse Electron Demand Diels-Alder Reaction (IEDDAR) between 1,2,4,5-tetrazine diester 6-1 (Scheme 6) and electron-rich dienophiles has proven to be an effective synthetic route toward substituted pyridazines. See e.g, Hamasaki, et al., J. Org. Chem., 2006, 71:185-193; Helm, et al., Angew. Chem. Int. Ed., 2005, 44:3889-3892.

Bak and Bcl-x_{L} belong to the Bcl-2 family of proteins, which regulate cell death through an intricate balance of homodimer and heterodimer complexes formed within this class of proteins. See Raff, Science, 1994, 264, 668-669; Chao &Korsmeyer, Annu. Rev. Immunol., 1998, 16, 395-419; Thompson, Science, 1995, 267:1456-1462; Rubin, et al., Curr. Biol., 1993, 3:391-394. Overexpression of anti-apoptotic proteins such as Bcl-x_{L} and Bcl-2 prevent cells from triggering programmed death pathways and has been linked to a variety of cancers. Bcl-2 protein plays a critical role in inhibiting anticancer drug-induced apoptosis, which is mediated by a mitochondria-dependent pathway that controls the release of cytochrome c from mitochondria through anion channels. Constitutive overexpression of Bcl-2 or unchanged expression after treatment with anticancer drugs confers drug resistance not only to hematologic malignancies but also to solid tumors. See Kim, et al. Cancer 2004, 101:2491-2502. A current strategy for developing new anticancer agents is to identify molecules that bind to the Bak-recognition site on Bcl-x_{L}, disrupting the complexation of the two proteins and therefore antagonizing Bcl-x_{L} function. See Kutzki, et al. J. Am. Chem. Soc. 2002, 124, 11, 832-11, 839. The structure determined by NMR spectroscopy (Sattler, et al. Science, 1997, 275:983-986) shows the 16 residue BH₃ domain peptide from Bak (aa 572 to 587, K_{d}z≈300 nM), having sequence GQVGRQLAIIGDDINR (SEQ ID NO:1), bound in a helical conformation to a hydrophobic cleft on the surface of Bcl-x_{L}, formed by the BH₁, BH₂, and BH₃ domains of the protein. The crucial residues for binding were shown by alanine scanning to be V574, L578, I581, and I585, which project in an i, i+4, i+7, i+11 arrangement from one face of the alpha-helix. The Bak peptide is a random coil in solution but adopts an alpha-helical conformation when complexed to Bcl-X_{L}. Studies utilizing stabilized helices of the Bak BH₃ domain have shown the importance of this conformation for tight binding. See Chin & Schepartz, Angew. Chem., 2001, 113:3922-3925; Angew. Chem. Int. Ed., 2001, 40:3806-3809.

### BRIEF SUMMARY OF THE INVENTION

There is provided a series of alpha-helix mimetic scaffolds. Certain of these scaffolds presents both a hydrophobic surface for recognition and a “wet edge” that is rich in hydrogen bond donors and acceptors. The terms “wet edge” and the like refer to hydrophilic surfaces as known in the art. Without wishing to be bound by any theory, it is believe that this structural feature enhances solubility and, during complexation with a target, the wet edge remains directed toward the solvent. Accordingly, there is little or no entropic penalty (or advantage) relating to solvation at the wet edge as it is minimally altered during docking of the helix mimetic. These scaffolds may be thought of as synthetic counterparts of amphiphilic alpha-helices, as known in the art. The alpha-helix mimetic scaffolds derive their activity from having a combination of, e.g., aliphatic and non-aliphatic functionalities. Preferred non-aliphatic functionalities employable with these scaffolds include, but are limited to, naturally occurring amino acid side chains or homologs thereof that are either aliphatic, polar, acidic, basic, or aromatic, or that contain a hydroxyl or thiol moiety.

In a first aspect, there is provided a nonpeptidic mimetic of an alpha-helix with structure of Formula (I)

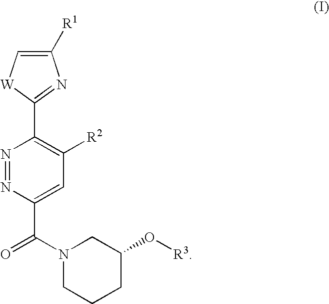

In Formula (I), W is —O— or —S—. R¹ and R² are independently hydrogen, substituted or unsubstituted alkyl, substituted or unsubstituted heteroalkyl, or a side chain of a naturally occurring amino acid, homolog thereof, or chemically protected analog thereof, optionally linked through an —O-ether linkage. R³ is substituted or unsubstituted alkyl, substituted or unsubstituted heteroalkyl, or a side chain of a naturally occurring amino acid, homolog thereof, or chemically protected analog thereof.

In another aspect, there is provided a nonpeptidic mimetic of an alpha-helix with structure of Formula (II):

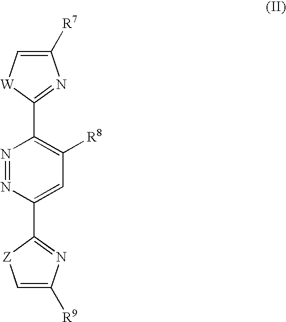

In Formula (II), W and Z are independently —O— or —S—. R⁷, R⁸ and R⁹ are independently hydrogen, substituted or unsubstituted alkyl, substituted or unsubstituted heteroalkyl, or a side chain of a naturally occurring amino acid, homolog thereof, or chemically protected analog thereof, optionally linked through an —O-ether linkage.

In another aspect, there is provided a method for synthesizing a compound of Formulae (I) or (II) and intermediates thereof.

In another aspect, there is provided a pharmaceutical composition comprising a therapeutically effective amount of a compound having the structure of either of Formulae (I) or (II) and a pharmaceutically acceptable carrier.

In another aspect, there is provided a method for disrupting a protein-protein interaction selected from the group consisting of Bak/Bcl-X_{L}, p53/HDM2, calmodulin/smooth muscle myosin light-chain kinase, and gp41 assembly. The method includes a step of contacting a compound of Formula (I) or Formula (II) with a sufficient amount to disrupt the protein-protein interaction.

In another aspect, there is provided a method for treating conditions and/or disorders mediated by the disruption of protein-protein interactions. The method includes the step of administering a compound of Formula (I) or (II) to a patient in need of treatment in an amount sufficient to disrupt the protein-protein interaction.

### DETAILED DESCRIPTION OF THE INVENTION

### I. Definitions

The following definitions are used herein.

Generally, reference to a certain element is meant to include all isotopes of that element. For example, if an substituent group is defined to include hydrogen, it also includes deuterium and tritium.

Alkyl groups include straight chain and branched alkyl groups having from 1 to about 20 carbon atoms, and typically from 1 to 12 carbons or, in some embodiments, from 1 to 8 carbon atoms. As used herein, “alkyl groups” include cycloalkyl groups as defined herein. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, isopentyl groups and the like. Representative substituted alkyl groups may be substituted one or more times with, for example, amino, carboxy, carboxamido, thio, hydroxy, alkoxy, and/or halo groups such as F, Cl, Br, and I groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which may be substituted with, for example, alkyl, alkoxy, amino, thio, hydroxy, cyano, and/or halo groups.

Cycloalkyl alkyl groups are alkyl groups as defined above in which a hydrogen or carbon bond of an alkyl group is replaced with a bond to a cycloalkyl group as defined above.

Alkenyl groups are straight chain, branched or cyclic alkyl groups having 2 to about 20 carbon atoms, and further including at least one double bond. In some embodiments alkenyl groups have from 2 to 12 carbons, or, typically, from 2 to 8 carbon atoms. Alkenyl groups include, for instance, vinyl, propenyl, 2-butenyl, 3-butenyl, isobutenyl, cyclohexenyl, cyclopentenyl, cyclohexadienyl, butadienyl, pentadienyl, and hexadienyl groups among others.

Alkynyl groups are straight chain or branched alkyl groups having 2 to about 20 carbon atoms, and further including at least one triple bond. In some embodiments alkynyl groups have from 2 to 12 carbons, or, typically, from 2 to 8 carbon atoms. Exemplary alkynyl groups include, but are not limited to, ethynyl, propynyl, and butynyl groups.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃. An “alkylene” and a “heteroalkylene,” alone or as part of another substituent, means a divalent radical derived from an alkyl or heteroalkyl, respectively.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

Aryl groups are cyclic aromatic hydrocarbons that do not contain heteroatoms. Thus, aryl groups include, but are not limited to, phenyl, azulenyl, heptalenyl, biphenylenyl, indacenyl, phenanthrenyl, triphenylenyl, pyrenyl, naphthacenyl, chrysenyl, biphenyl, anthracenyl, and naphthenyl groups. Although the phrase “aryl groups” includes groups containing fused rings, such as fused aromatic-aliphatic ring systems (e.g., indanyl, tetrahydronaphthyl, and the like) and fused aromatic-unsaturated ring systems (e.g., indenyl, fluorenyl, and the like). It does not include aryl groups that have other groups, such as alkyl or halo groups, bonded to one of the ring members. Rather, groups such as tolyl are referred to as substituted aryl groups. Representative substituted aryl groups may be mono-substituted or substituted more than once, such as, but not limited to, 2-, 3-, 4-, 5-, or 6-substituted phenyl or naphthyl groups, which may be substituted with groups including, but not limited to, amino, nitro, carboxy, carboxamido, hydroxy, thio, alkoxy, alkyl, cyano, and/or halo.

Aralkyl groups are alkyl groups as defined above in which a hydrogen or carbon bond of an alkyl group is replaced with a bond to an aryl group as defined above. Representative aralkyl groups include benzyl groups and fused (cycloalkylaryl)alkyl groups such as 4-ethyl-indanyl.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. The phrase “heterocyclyl group” includes mono-, bi-, and polycyclic ring systems. Heterocyclyl groups thus include fused ring species including those comprising fused aromatic and non-aromatic groups. The phrase also includes bridged polycyclic ring systems containing one or more heteroatoms such as, but not limited to, quinuclidyl. However, the phrase does not include heterocyclyl groups that have other groups, such as alkyl or halo groups, bonded to one of the ring members. Rather, these are referred to as “substituted heterocyclyl groups”. Heterocyclyl groups include, but are not limited to, pyrrolidinyl, piperidinyl, piperazinyl, morpholinyl, pyrrolyl, pyrazolyl, triazolyl, imidazolyl, imidazolidinyl, tetrazolyl, oxazolyl, oxazolinyl, oxazolidinyl, isoxazolyl, isoxazolinyl, isoxazolidinyl, thiazolyl, pyridinyl, pyridazinyl, pyrazinyl, pyrimidinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups may be mono-substituted or substituted more than once, such as, but not limited to, pyridazinyl, pyridinyl, oxazolidinyl, oxazolinyl, or oxazolyl groups, which are 2-, 3-, 4-, 5-, or 6-substituted, or disubstituted with groups including, but not limited to, amino, hydroxyl, thio, alkoxy, alkyl, cyano, and/or halo.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, imidazolyl, triazolyl, tetrazolyl, oxazolyl, oxazolinyl, oxazolidinyl, isoxazolyl, isoxazolinyl, isoxazolidinyl, thiazolyl, pyridinyl, pyridazinyl, pyrazinyl, pyrimidinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydroindolyl, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups including, but not limited to, amino, alkoxy, alkyl, thio, hydroxy, cyano, and/or halo.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridin-3-yl methyl, tetrahydrofuran-2-yl ethyl, indol-2-yl methyl, and indol-2-yl propyl.

Heteroaralkyl groups are alkyl groups as defined above in which a hydrogen or carbon bond of an alkyl group is replaced with a bond to a heteroaryl group as defined above.

In general, “substituted” refers to a group as defined above in which one or more bonds to a hydrogen atom contained therein are replaced by a bond to non-hydrogen atoms such as, but not limited to, an alkyl, cycloalkyl, heteroalkyl, or heterocycloalkyl; a halogen atom such as F, Cl, Br, and I; an oxygen atom in groups such as hydroxyl groups, alkoxy groups, aryloxy groups, aralkoxy groups, and ester groups; a sulfur atom in groups such as thiol groups, alkyl and aryl sulfide groups, sulfone groups, sulfonyl groups, sulfonamide, and sulfoxide groups; a nitrogen atom in groups such as nitro groups, amines, amides, alkylamines, dialkylamines, arylamines, alkylarylamines, diarylamines, N-oxides, imides, ureas, guanidines, amidines and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups and also substituted cycloalkyl groups and others also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles.

Substituted ring systems such as, but not limited to, substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with alkyl groups, alkenyl groups, or alkynyl groups as defined above.

The term “protected” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W.; Wuts, P. G. M., John Wiley & Sons, New York, N.Y., (3rd Edition, 1999) which can be added or removed using the procedures set forth therein. Examples of protected hydroxyl groups include, but are not limited to, silyl ethers such as those obtained by reaction of a hydroxyl group with a reagent such as, but not limited to, t-butyldimethyl chlorosilane, trimethylchlorosilane, triisopropylchlorosilane, triethylchlorosilane; substituted methyl and ethyl ethers such as, but not limited to methoxymethyl ether, methylthiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; imides, such as phthalimide, and dithiosuccinimide; and others. Examples of protected sulfhydryl groups include, but are not limited to, thioethers such as S-benzyl thioether, and S-4-picolyl thioether; substituted S-methyl derivatives such as hemithio, dithio and aminothio acetals; and others. A “chemically protected analog,” as used herein, refers to a protected a compound described herein that is protected. The “chemically protected analog” may have one or a plurality of protecting groups.

Side chains of amino acids are the groups attached to the alpha carbon of alpha-amino acids. For example the side chains of glycine, alanine, and phenylalanine are hydrogen, methyl, and benzyl, respectively. The side chains may be of any naturally occurring or synthetic alpha amino acid. Naturally occurring alpha amino acids include those found in naturally occurring peptides, proteins, hormones, neurotransmitters, and other naturally occurring molecules. Synthetic alpha amino acids include any non-naturally occurring amino acid known to those of skill in the art. Representative amino acids include, but are not limited to, glycine, alanine, serine, threonine, arginine, lysine, ornithine, aspartic acid, glutamic acid, asparagine, glutamine, phenylalanine, tyrosine, tryptophan, leucine, valine, isoleucine, cysteine, methionine, histidine, 4-trifluoromethyl-phenylalanine, 3-(2-pyridyl)-alanine, 3-(2-furyl)-alanine, 2,4-diaminobutyric acid, and the like.

Pharmaceutically acceptable salts include a salt with an inorganic base, organic base, inorganic acid, organic acid, or basic or acidic amino acid. As salts of inorganic bases, the invention includes, for example, alkali metals such as sodium or potassium, alkali earth metals such as calcium and magnesium or aluminum, and ammonia. As salts of organic bases, the invention includes, for example, trimethylamine, triethylamine, pyridine, picoline, ethanolamine, diethanolamine, triethanolamine. As salts of inorganic acids, the instant invention includes, for example, hydrochloric acid, boric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, lactic acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Certain compounds within the scope of Formulae (I) and (II) are derivatives customarily referred to as prodrugs. The expression “prodrug” denotes a derivative of a known direct acting drug, e.g. esters and amides, which derivative has enhanced delivery characteristics and therapeutic value as compared to the drug, and is transformed into the active drug by an enzymatic or chemical process; see Notari, Methods in Enzymology, 1985, 112:309-323; Bodor, Drugs of the Future, 1981, 6:165-182; Bundgaard, in Design of Prodrugs (H. Bundgaard, ed.), Elsevier, N.Y. (1985); Goodman & Gilmans, The Pharmacological Basis of Therapeutics, 8th ed., McGraw-Hill, Int. Ed. 1992. The preceding references are hereby incorporated by reference in their entirety and for all purposes.

Tautomers refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, ketones are typically in equilibrium with their enol forms. Thus, ketones and their enols are referred to as tautomers of each other. As readily understood by one skilled in the art, a wide variety of functional groups and other structures may exhibit tautomerism, and all tautomers of compounds having Formula I or Formula IA are within the scope of the present invention.

Compounds of the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

“Treating” within the context of the instant invention, means an alleviation, in whole or in part, of symptoms associated with a disorder or disease, or halt of further progression or worsening of those symptoms, or prevention or prophylaxis of the disease or disorder. Similarly, as used herein, a “therapeutically effective amount” of a compound of the invention refers to an amount of the compound that alleviates, in whole or in part, symptoms associated with a disorder or disease, or halts of further progression or worsening of those symptoms, or prevents or provides prophylaxis for the disease or disorder. Treatment may also include administering the pharmaceutical Formulations of the present invention in combination with other therapies. For example, the compounds of the invention can also be administered in conjunction with other therapeutic agents against bone disease or agents used for the treatment of metabolic disorders.

Naturally occurring polar amino acids include arginine, asparagine, aspartic acid, glutamic acid, glutamine, histidine, lysine, serine, threonine, and tyrosine. Naturally occurring acidic amino acids include aspartic acid and glutamic acid. Naturally occurring basic amino acids include arginine, histidine, and lysine. Naturally occurring aromatic amino acids include phenylalanine, tryptophan, and tyrosine. Naturally occurring amino acids that contain a hydroxyl or thiol moiety include cysteine, methionine, and threonine. Naturally occurring aliphatic amino acids include alanine, leucine, isoleucine and valine. An analog of a naturally occurring amino acid is a structural derivative of the naturally occurring amino acid that differs from it by a single element or by a substitution of a functional moiety within its side chain with a homolog of such functional moiety. A functional moiety is a specific group of atoms within a molecule that is responsible for chemical characteristic of such molecule, as known in the art.

### II. Compositions

In one aspect, there is provided a nonpeptidic mimetic of an alpha-helix with structure of Formula (I)

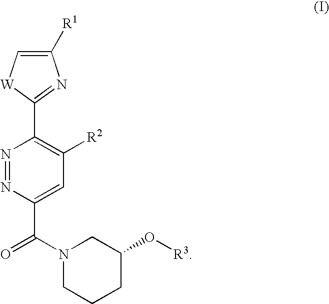

In Formula (I), W is —O— or —S—. R¹ and R² are independently hydrogen, substituted or unsubstituted alkyl, substituted or unsubstituted heteroalkyl, or a side chain of a naturally occurring amino acid, homolog thereof, or chemically protected analog thereof, optionally linked through an —O-ether linkage. R³ is substituted or unsubstituted alkyl, substituted or unsubstituted heteroalkyl, or a side chain of a naturally occurring amino acid, homolog thereof, or chemically protected analog thereof “Homolog” refers in the customary sense to elongation (or shortening) of a substituent described herein by insertion (or deletion) of one or more hydrocarbon functionalities. For example, diaminopropionic acid, diaminobutyric acid, ornithine, lysine, and homolysine form a homologous series for lysine. The term “chemically protected analog” refers to compounds having chemical protecting groups. Exemplary chemical protecting groups are well known in the art and include, but not limited to, Boc, FMoc, benzyl (Bn), tert-Bu, trityl (—CPh₃), and the like. Where R¹ and/or R² are “optionally linked through an —O-ether linkage”, it is meant that R¹ and/or R² are indirectly attached to the remainder of the molecule via a divalent oxygen linker (i.e. an ether).

Thus, in some embodiments, R¹ and/or R² are -L¹-R^{1A} and/or -L²-R^{2A}, respectively, wherein R^{1A} and/or R^{2A} are independently hydrogen, substituted or unsubstituted alkyl, substituted or unsubstituted heteroalkyl, or a side chain of a naturally occurring amino acid, homolog thereof, or chemically protected analog thereof. L¹ and L² are independently a bond or —O—. In some embodiments, the definitions of R¹ and R² described herein (without regard to the optionally linkage through an —O-ether linkage) are equally applicable to R^{1A} and R^{2A}, respectively. Thus, in some embodiments, the compound has the structure of Formula (IA):

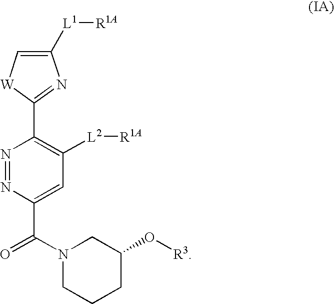

In Formula (IA), R^{1A} and R^{2A} are, respectively, defined the same as R¹ and R² as described herein (without regard to the portions of the R¹ and R² definitions relating to the optional —O-ether linkage). L¹ and L² are independently a bond or —O—.

Without wishing to be bound by any theory, it is believed that compounds of Formula (I) useful in the methods described herein mimic at least part of the structure of an alpha-helical segment of a protein involved in a protein-protein interaction. Accordingly, R¹ may mimic the side chain of a residue protruding away from the backbone of an alpha helical segment at an arbitrary residue (with index “i”) within the protein sequence, R² may mimic the side chain of residue i+3 or i+4 within the sequence, and/or R³ may mimic the side chain of residue i+7 within the sequence. The term “i+x” in the context of proteins refers, in the customary sense, to a residue having a position in the primary sequence of the protein which is “x” residues away from a residue “i.” Alternatively, R³ may mimic the side chain of a residue protruding away from the backbone of an alpha helical segment at a residue “i” within the protein sequence, R² may mimic the side chain of residue i+3 or i+4 within the sequence, and/or R¹ may mimic the side chain of residue i+7 within the sequence. The terms “mimic the chain side of a residue” and the like in this context refer to a conformation of a compound of Formula (I) wherein the distance between R¹ and R², and the distance between R² and R³ are approximately the distances between the side chains of residues in an alpha-helix at the i to i+3/i+4, and i to i+7 positions, as known in the art. In some embodiments, R¹ corresponds to the “i” position of an alpha-helix; R² corresponds to the i+3 or i+4 position of an alpha-helix; and R³ corresponds to the i+7 position of an alpha-helix. In some embodiments, R³ corresponds to the “i” position of an alpha-helix; R² corresponds to the i+3 or i+4 position of an alpha-helix; and R¹ corresponds to the i+7 position of an alpha-helix.

In some embodiments, one and only one of R¹, R^{1A}, R², R^{2A} and R³ is hydrogen. In some embodiments, L¹ and L² are bonds. In some embodiments, W is —O—.

In some embodiments, R¹, R^{1A}, R², R^{2A} and R³ are independently hydrogen, R⁴-substituted or unsubstituted alkyl, R⁴-substituted or unsubstituted heteroalkyl, or a chemically protected analog thereof. R⁴ is independently halogen, —CN, —CF₃, —OH, —NH₂, —SO₂, —COOH, R⁵-substituted or unsubstituted alkyl, R⁵-substituted or unsubstituted heteroalkyl, R⁵-substituted or unsubstituted cycloalkyl, R⁵-substituted or unsubstituted heterocycloalkyl, R⁵-substituted or unsubstituted aryl, or R⁵-substituted or unsubstituted heteroaryl. R⁵ is independently halogen, —NO₂, —CN, —CF₃, —OH, —NH₂, —SO₂, —COOH, R⁶-substituted or unsubstituted alkyl, R⁶-substituted or unsubstituted heteroalkyl, R⁶-substituted or unsubstituted cycloalkyl, R⁶-substituted or unsubstituted heterocycloalkyl, R⁶-substituted or unsubstituted aryl, or R⁶-substituted or unsubstituted heteroaryl. R⁶ is independently halogen, —NO₂, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In some embodiments, R¹, R^{1A}, R² and R^{2A} are independently hydrogen, R⁴-substituted or unsubstituted C₁-C₁₀ (e.g., C₁-C₆) alkyl, R⁴-substituted or unsubstituted 2 to 10 membered (e.g., 2 to 6 membered) heteroalkyl, or a chemically protected analog thereof.

In some embodiments, R¹, R^{1A}, R², R^{2A} and R³ are each independently —(C₂-C₉ alkyl), —CH₂(C₃-C₈ cycloalkyl), —CH₂(C₆-C₁₀ aryl), or a side chain of a naturally occurring amino acid or homolog thereof. In some embodiments, at least one of the following provisos apply: 1) at least one of R¹, R^{1A}, R², R^{2A} and R³ is a side chain of a naturally occurring polar amino acid or homolog thereof; 2) at least one of R¹, R^{1A}, R², R^{2A} and R³ is a side chain of a naturally occurring acidic amino acid or homolog thereof; 3) at least one of R¹, R^{1A}, R², R^{2A} and R³ is a side chain of a naturally occurring basic amino acid or homolog thereof; 4) at least one of R¹, R^{1A}, R², R^{2A} and R³ is a side chain of a naturally occurring aromatic amino acid or homolog thereof; 5) at least one of R¹, R^{1A}, R², R^{2A} and R³ is a side chain of a naturally occurring amino acid containing a hydroxyl or thiol moiety or homolog thereof; or 6) at least one of R¹, R^{1A}, R², R^{2A} and R³ is a side chain of a naturally occurring aliphatic amino acid or homolog thereof.

In some embodiments, R² is -(substituted or unsubstituted C₁-C₉ alkyl), —CH₂-(substituted or unsubstituted C₃-C₈ cycloalkyl), —CH₂-(substituted or unsubstituted C₆-C₁₀ aryl), —(CH₂)₂—SCPh₃, —(CH₂)₂—SMe, —(CH₂)₃—NBoc, —(CH₂)₂—NBoc, —(CH₂)₂—CO₂-^{t}Bu,

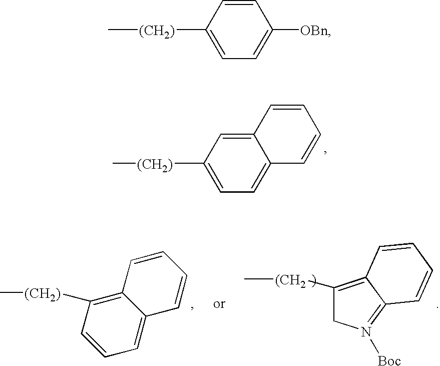

In other embodiments, R² is -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, —(CH₂)₂—CO₂-^{t}Bu,

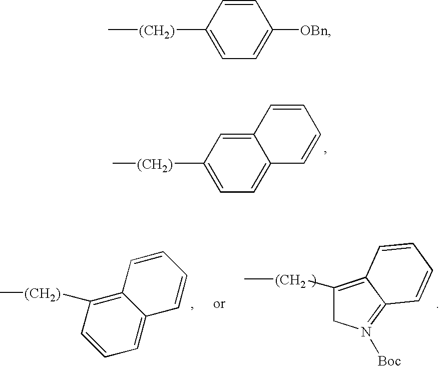

In some embodiments, R² is -(substituted or unsubstituted C₁-C₉ alkyl), —CH₂-(substituted or unsubstituted C₃-C₈ cycloalkyl), —CH₂-(substituted or unsubstituted C₆-C₁₀ aryl), —(CH₂)₂—SH, —(CH₂)₂—SMe, —(CH₂)₃—NH₂, —(CH₂)₂—NH₂, —(CH₂)₂—COOH,

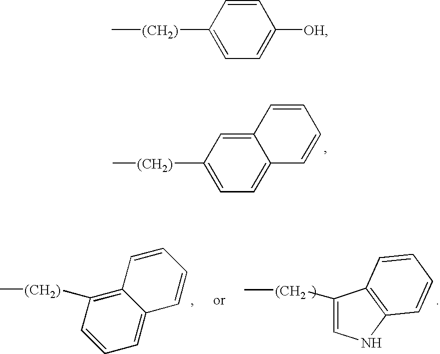

In other embodiments, R² is -(unsubstituted C₁-C₉ alkyl), —CH₂-(unsubstituted C₃-C₈ cycloalkyl), —CH₂-(unsubstituted C₆-C₁₀ aryl), —(CH₂)₂—SH, —(CH₂)₂—SMe, —(CH₂)₃—NH₂, —(CH₂)₂—NH₂, —(CH₂)₂—COOH,

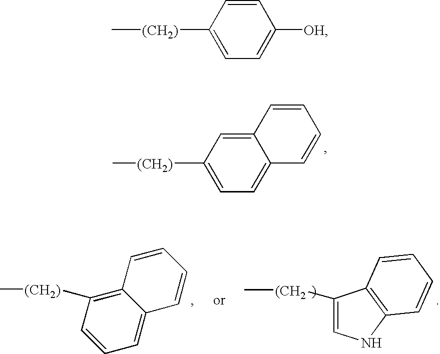

In some embodiments, R² is —(C₁-C₉ substituted or unsubstituted alkyl), substituted —CH₂-(substituted or unsubstituted C₃-C₈ cycloalkyl), —CH₂-(substituted or unsubstituted C₆-C₁₀ aryl), —(CH₂)₂—SCPh₃, —(CH₂)₂—SMe, —(CH₂)₃—NBoc, —(CH₂)₂—NBoc, or —(CH₂)₂—CO₂-^{t}Bu.

In some embodiments, R² is -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, or —(CH₂)₂—CO₂-^{t}Bu.

In some embodiments, the side chains of the naturally occurring amino acid with respect to R¹, R^{1A}, R², R^{2A} and R³ are independently —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), —OC(O)—(C₁-C₆ alkyl) or homolog thereof.

Some species of an embodiment with acidic amino acid side chains are illustrated as follows:

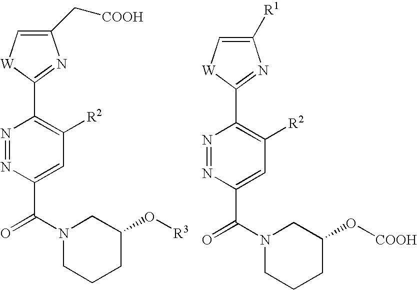

In other embodiments, at least one of R¹, R^{1A}, R², R^{2A} and R³ is a side chain of a naturally occurring basic amino acid or homolog thereof. A species of this embodiment with a basic amino acid side chain is illustrated as follows:

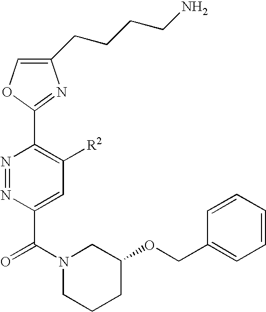

In some embodiments, at least one R¹, R^{1A}, R², R^{2A} and R³ is a side chain of a naturally occurring aromatic amino acid or homolog thereof. In some embodiments, at least one of R¹, R^{1A}, R², R^{2A} and R³ is a side chain of a naturally occurring amino acid containing a hydroxyl or thiol moiety, or homolog thereof.

In some embodiments, a compound is provided having the structure of Formula (IB), wherein the substituents are as defined for Formulae (I) and (IA).

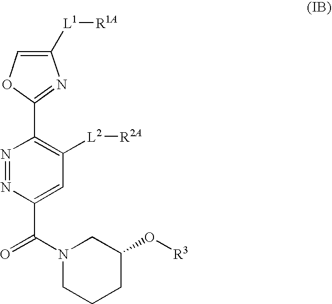

In another aspect, there is provided a nonpeptidic mimetic of an alpha-helix with structure of Formula (II):

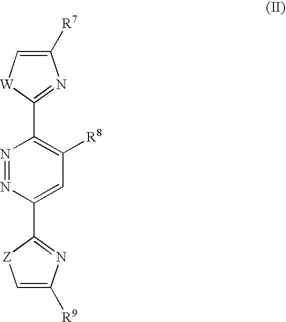

In Formula (II), W and Z are independently —O— or —S—. R⁷, R⁸ and R⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a side chain of a naturally occurring amino acid, homolog thereof, or chemically protected analog thereof, optionally linked through an —O-ether linkage. In some embodiments, R⁷, R⁸ and R⁹ are independently hydrogen, substituted or unsubstituted alkyl, substituted or unsubstituted heteroalkyl, or a side chain of a naturally occurring amino acid, homolog thereof, or chemically protected analog thereof, optionally linked through an —O-ether linkage. Where R⁷, R⁸ and/or R⁹ are “optionally linked through an —O-ether linkage”, it is meant that R⁷, R⁸ and/or R⁹ is indirectly attached to the remainder of the molecule via a divalent oxygen linker (i.e. an ether).

Thus, in some embodiments, R⁷, R⁸ and/or R⁹ are -L⁴-R^{7A}, -L⁵-R^{8A} and/or -L⁶-R^{9A}, respectively, wherein R^{7A}, R^{8A} and/or R^{9A} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a side chain of a naturally occurring amino acid, homolog thereof, or chemically protected analog thereof. In some embodiments, R⁷, R⁸ and/or R⁹ are -L⁴-R^{7A}, -L⁵-R^{8A} and/or -L⁶-R^{9A}, respectively, wherein R^{7A}, R^{8A} and/or R^{9A} are independently hydrogen, substituted or unsubstituted alkyl, substituted or unsubstituted heteroalkyl, or a side chain of a naturally occurring amino acid, homolog thereof, or chemically protected analog thereof. L⁴, L⁵ and L⁶ are independently a bond or —O—. In some embodiments, the definitions of R⁷, R⁸ and/or R⁹ described herein (without regard to the optionally linkage through an ether linkage) are equally applicable to R^{7A}, R^{8A} and/or R^{9A}, respectively. Thus, in some embodiments, the compound has the formula:

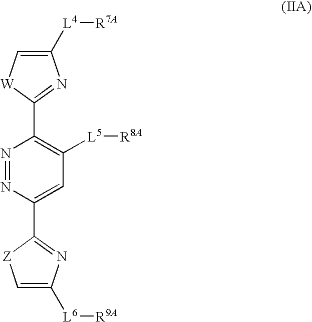

In some embodiments, R⁷ corresponds to the i position of an alpha-helix; R⁸ corresponds to the i+3/i+4 position of an alpha-helix; and R⁹ corresponds to the i+11 position of an alpha-helix. In some embodiments, R⁹ corresponds to the i position of an alpha-helix; R⁸ corresponds to the i+7 position of an alpha-helix; and R⁷ corresponds to the i+11 position of an alpha-helix.

In some embodiments, one and only one of R⁷, R⁸ and R⁹ is hydrogen.

In some embodiments, R⁷, R^{7A}, R⁸, R^{8A}, R⁹ and R^{9A} are independently hydrogen, R¹⁰-substituted or unsubstituted alkyl, R¹⁰-substituted or unsubstituted heteroalkyl, R¹⁰-substituted or unsubstituted cycloalkyl, R¹⁰-substituted or unsubstituted heterocycloalkyl, R¹⁰-substituted or unsubstituted aryl, R¹⁰-substituted or unsubstituted heteroaryl, or a chemically protected analog thereof. In some embodiments, R⁷, R^{7A}, R⁸, R^{8A}, R⁹ and R^{9A} are independently hydrogen, R¹⁰-substituted or unsubstituted alkyl, R¹⁰-substituted or unsubstituted heteroalkyl, or a chemically protected analog thereof. R¹⁰ is independently halogen, —CN, —CF₃, —OH, —NH₂, —SO₂, —COOH, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. R¹¹ is independently halogen, —NO₂, —CN, —CF₃, —OH, —NH₂, —SO₂, —COOH, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl. R¹² is independently halogen, —NO₂, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In some embodiments, R⁷, R^{7A}, R⁸, R^{8A}, R⁹ and R^{9A} are independently hydrogen, R¹⁰-substituted or unsubstituted C₁-C₁₀ (e.g., C₁-C₆) alkyl, R¹⁰-substituted or unsubstituted 2 to 10 membered (e.g., 2 to 6 membered) heteroalkyl, or a chemically protected analog thereof.

In some embodiments, R⁷, R^{7A}, R⁸, R^{8A}, R⁹ and R^{9A} are each independently —(C₂-C₉ substituted or unsubstituted alkyl), —CH₂(C₃-C₈ substituted or unsubstituted cycloalkyl), —CH₂(C₆-C₁₀ substituted or unsubstituted aryl), a side chain of a naturally occurring amino acid or a homolog thereof. In some embodiments, R⁷, R^{7A}, R⁸, R^{8A}, R⁹ and R^{9A} are each independently —(C₂-C₉ unsubstituted alkyl), —CH₂(C₃-C₈ unsubstituted cycloalkyl), —CH₂(C₆-C₁₀ unsubstituted aryl), a side chain of a naturally occurring amino acid or a homolog thereof. In some embodiments, W and Z are each independently selected from the group consisting of —O— and —S—. In some embodiments, W and Z are —O—.

In some embodiments, at least one of the following provisos apply: 1) at least one of R⁷, R^{7A}, R⁸, R^{8A}, R⁹ and R^{9A} is a side chain of a naturally occurring polar amino acid or homolog thereof; 2) at least one of R⁷, R^{7A}, R⁸, R^{8A}, R⁹ and R^{9A} is a side chain of a naturally occurring acidic amino acid or homolog thereof; 3) at least one of R⁷, R^{7A}, R⁸, R^{8A}, R⁹ and R^{9A} is a side chain of a naturally occurring aromatic amino acid or homolog thereof; 4) at least one of R⁷, R^{7A}, R⁸, R^{8A}, R⁹ and R^{9A} is a side chain of a naturally occurring amino acid containing a hydroxyl or thiol moiety or homolog thereof; 5) at least one of R⁷, R^{7A}, R⁸, R^{8A}, R⁹ and R^{9A} is a side chain of a naturally occurring basic amino acid or homolog thereof; or 6) at least one of R⁷, R^{7A}, R⁸, R^{8A}, R⁹ and R^{9A} is a side chain of a naturally occurring aliphatic amino acid or homolog thereof.

In some embodiments, R⁸ is -(substituted or unsubstituted C₁-C₉ alkyl), —CH₂-(substituted or unsubstituted C₃-C₈ cycloalkyl), —CH₂-(substituted or unsubstituted C₆-C₁₀ aryl), —(CH₂)₂—SCPh₃, —(CH₂)₂—SMe, —(CH₂)₃—NBoc, —(CH₂)₂—NBoc, —(CH₂)₂—CO₂-^{t}Bu,

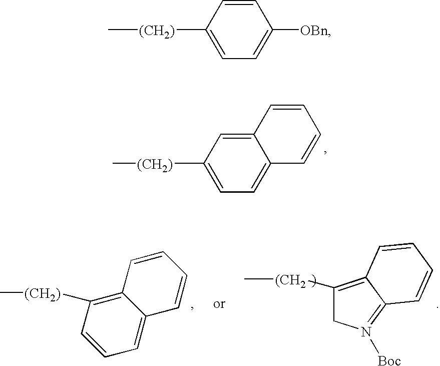

In other embodiments, R⁸ is -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, —(CH₂)₂—CO₂-^{t}Bu,

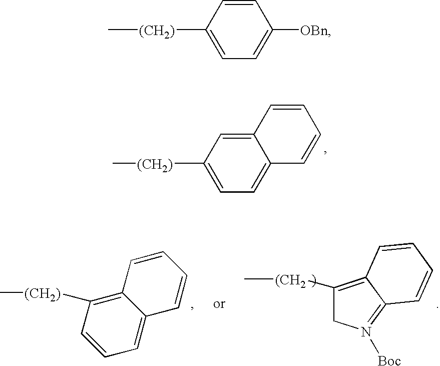

In some embodiments, R⁸ is -(substituted or unsubstituted C₁-C₉ alkyl), —CH₂-(substituted or unsubstituted C₃-C₈ cycloalkyl), —CH₂-(substituted or unsubstituted C₆-C₁₀ aryl), —(CH₂)₂—SH, —(CH₂)₂—SMe, —(CH₂)₃—NH₂, —(CH₂)₂—NH₂, —(CH₂)₂—COOH,

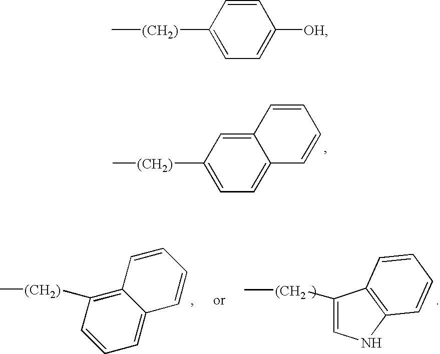

In other embodiments, R⁸ is -(unsubstituted C₁-C₉ alkyl), —CH₂-(unsubstituted C₃-C₈ cycloalkyl), —CH₂-(unsubstituted C₆-C₁₀ aryl), —(CH₂)₂—SH, —(CH₂)₂—SMe, —(CH₂)₃—NH₂, —(CH₂)₂—NH₂, —(CH₂)₂—COOH,

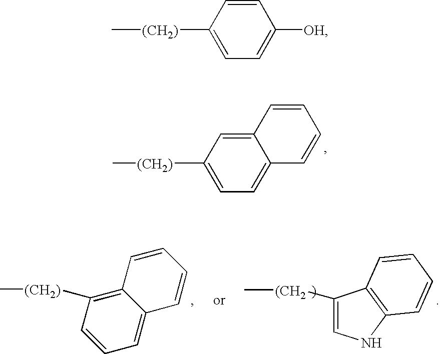

In some embodiments, R⁸ is —(C₁-C₉ substituted or unsubstituted alkyl), substituted —CH₂-(substituted or unsubstituted C₃-C₈ cycloalkyl), —CH₂-(substituted or unsubstituted C₆-C₁₀ aryl), —(CH₂)₂—SCPh₃, —(CH₂)₂—SMe, —(CH₂)₃—NBoc, —(CH₂)₂—NBoc, or —(CH₂)₂—CO₂-^{t}Bu.

In some embodiments, R⁸ is -(unsubstituted C₁-C₉ alkyl), —CH₂-(unsubstituted C₃-C₈ cycloalkyl), —CH₂-(unsubstituted C₆-C₁₀ aryl), —(CH₂)₂—SCPh₃, —(CH₂)₂—SMe, —(CH₂)₃—NBoc, —(CH₂)₂—NBoc, or —(CH₂)₂—CO₂-^{t}Bu.

In some embodiments, the side chain of the naturally occurring amino acid with respect to each of R⁷, R^{7A}, R⁸, R^{8A}, R⁹ and R^{9A} is independently —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), —OC(O)—(C₁-C₆ alkyl) or homolog thereof. Species of an embodiment with acidic amino acid side chains are illustrated as follows:

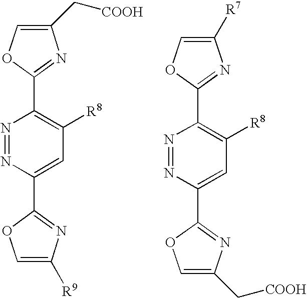

In another embodiment, at least one of R⁷, R^{7A}, R⁸, R^{8A}, R⁹ and R^{9A} is a side chain of a naturally occurring basic amino acid or homolog thereof. A species of this embodiment with a basic amino acid side chain is represented as follows:

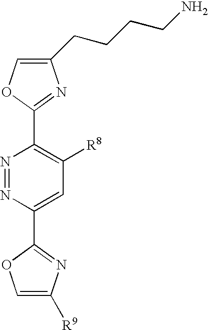

In some embodiments, there is provided a compound with the structure of Formula (IIB), wherein the substituents are as defined for Formulae (II) and (IIA).

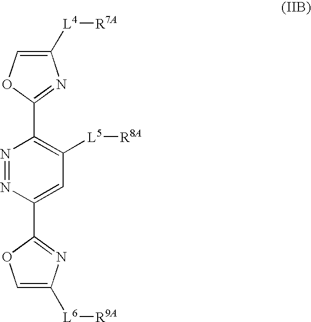

Exemplary compounds having the structure of Formula (II) are represented, but not limited to, the following:

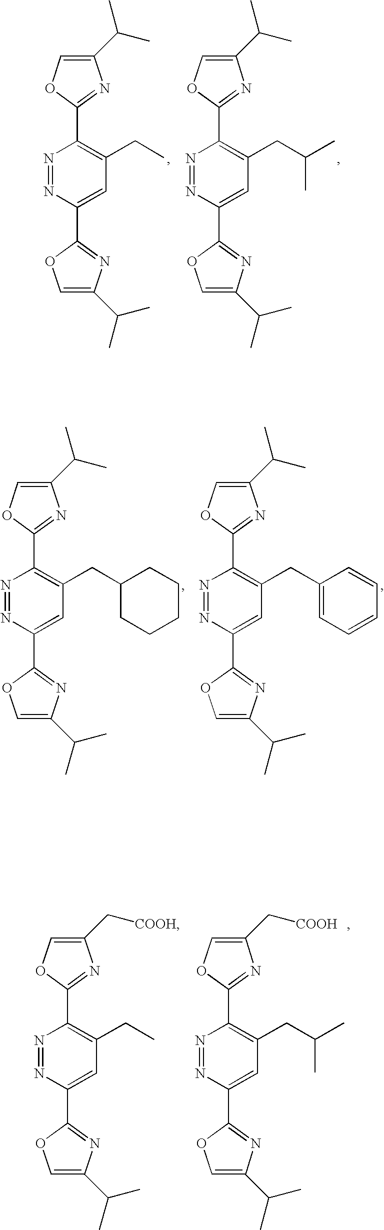

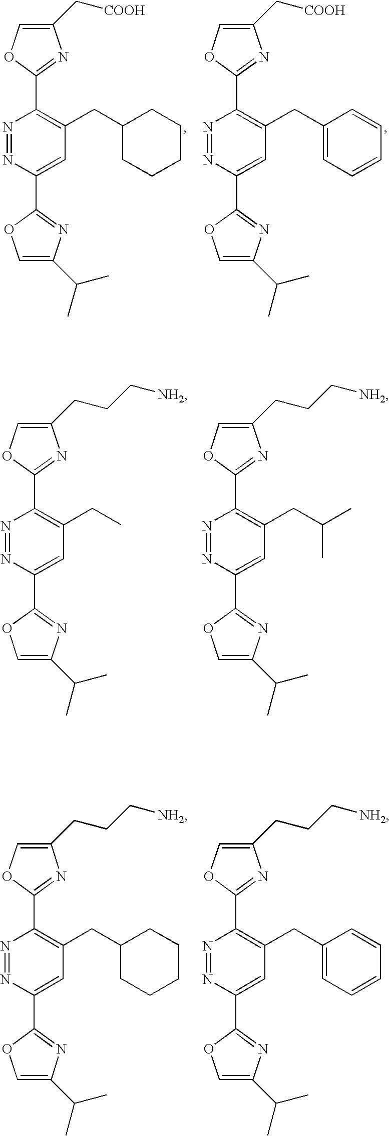

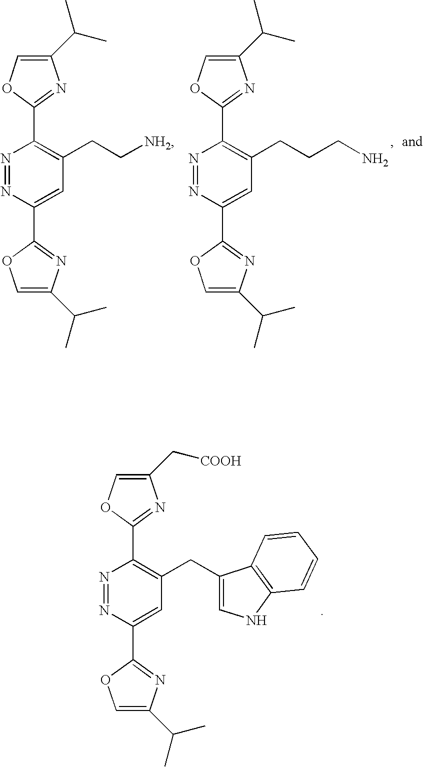

### III. Exemplary Syntheses

General retrosynthesis of oxazole-pyridazine-piperidines. A general retrosynthetic approach to synthesis of oxazole-pyridazine-piperidine scaffolds disclosed herein is provided in Scheme 1 following. The major disconnections from the final oxazole-pyridazine-piperidine compound 4.6 are made at the amide bonds to give a pyridazine diester (4.9), an amino-alcohol (4.8) and a piperidine (4.10). As known in the art, such synthesis is modular, as each piece can be synthesized separately and attached in sequence. Many amino-alcohols are commercially available, or available to one of skill in the art, bearing either natural amino acid side chains or homologs thereof

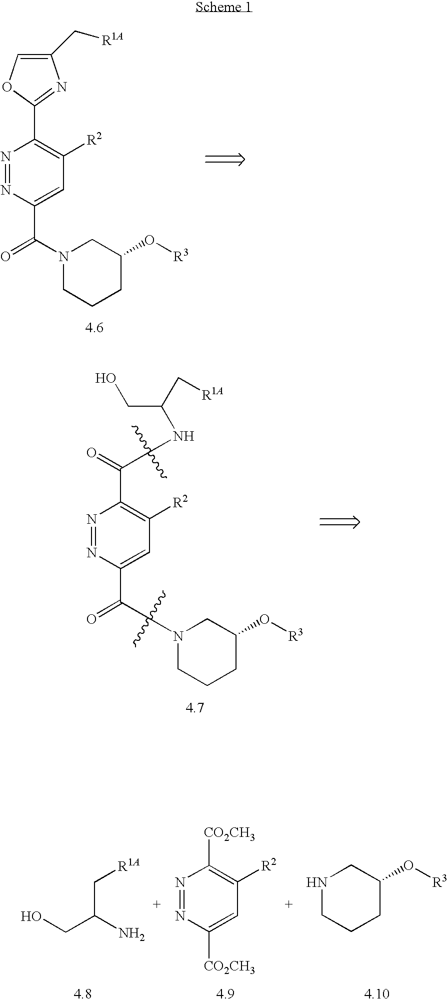

General retrosynthesis of oxazole-pyridazine-oxazoles. A general retrosynthetic approach to synthesis of oxazole-pyridazine-oxazole scaffolds illustrated herein is provided in Scheme 2 following. The major disconnections from the final oxazole-pyridazine-oxazole compound are made at the amide bonds to give a pyridazine diester (4.9) and the amino-alcohols (4.8′ and 4.8″).

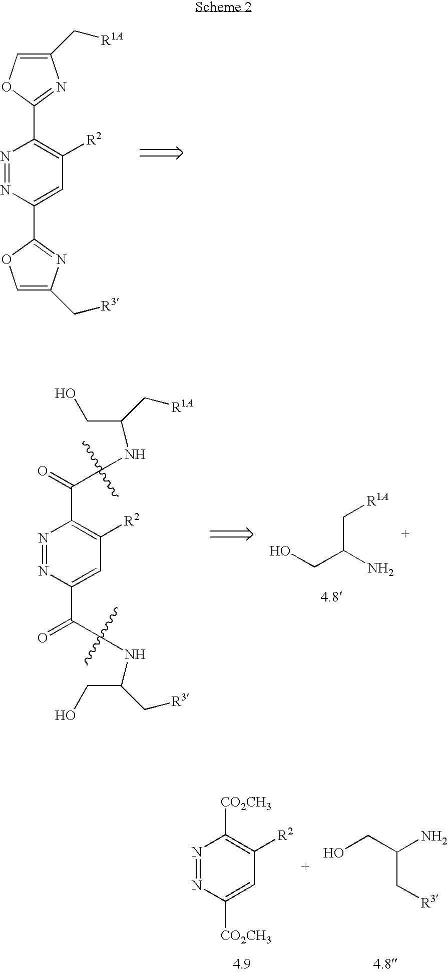

General retrosynthesis of substituted pyridazines. A general retrosynthetic approach for the synthesis of the pyridazine element of compounds disclosed herein is provided in Scheme 3 following. As described herein and known in the art, the pyridazine ring is readily available from the Inverse Electron Demand Diels-Alder (IEDDA) reaction of known dimethyl-1,2,4,5-tetrazine-dicarboxylate and a suitable dienophile. See e.g., Boger, et al., Org. Synth., 1992, 70:79-87.

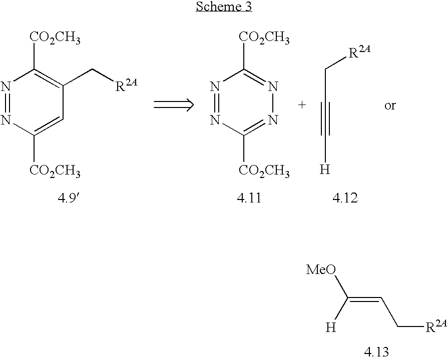

General synthesis of substituted pyridazines. Typically, the Inverse Electron Demand Diels Alder reaction is performed in an organic solvent such as diethyl ether, pentane, toluene, chloroform, dioxane, carbon tetrachloride, nitrobenzene, dichloromethane, ethyl acetate, THF, benzene, acetonitrile, dimethyl ether, 1,2-dichloroethane, xylene, acetone, chlorobenzene, DMSO, methanol, mesitylene and the like, or mixtures thereof. The reaction can be performed at room temperature, or can be heated to between 80-140° C. Typically, the reaction is performed at 80-100° C. Scheme 4 following illustrates a typical procedure to obtain intermediates in the synthesis of compounds of Formulae (I) or (II). The starting dimethyl 1,2,4,5-tetrazine-dicarboxylate is prepared by known methods. See Boger et al., Org. Synth. Id.; Spencer et al., J. Chem. Phy., 1961, 35:1939; Sauer, et al., Chem. Ber., 1965, 98:1435.

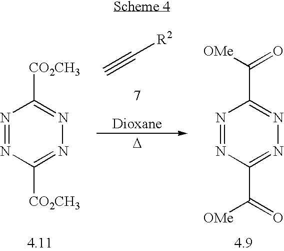

The inverse electron demand Diels Alder reaction with 1,2,4,5-tetrazine can also be performed with alkenes substituted with a leaving group, such as O-TMS, —SO-phenyl, morpholino, or pyrrolidino and the like (see Boger, Tetrahedron, 1983, Id.), by essentially the same procedures as described for Scheme 4.

Additionally, enolates, which can be generated in situ by reaction with aldehydes in the presence of a base (such as KOH, NaOH, LiOH, KOtBu, NaOMe, NaOEt, NaH and the like) can be used as reaction partners in the Diels Alder reaction. Scheme 5 following shows an additional application, in which the Diels Alder reaction is performed with dihydrofuran or dihydropyran derivatives, yielding compounds useful in the synthesis of compounds of Formulae (I) or (II), wherein n is 1, 2 or 3.

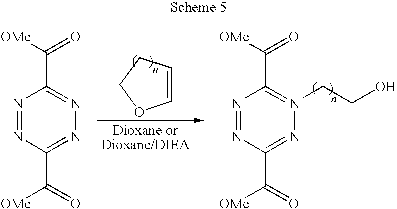

Further synthetic utility is available via the IEDDA reaction of dihydrofuran, dihydropyran and substituted derivatives thereof with the tetrazine. For example, as shown in Scheme 6 following, the diester 6-1 (Boger, et al., Org. Synth., 1992, Id.; Naud, Synlett 2004:2836-2837) can be reacted with the commercially available 2-methoxy-3,4-dihydro-2H-pyrane to give pyridazine 6-4 in good yield. Subsequent treatment under standard Fischer indole synthesis (see e.g., Hutchins & Chapman, Tetrahedron Lett., 1996, 37:4869-4872) conditions leads predominantly to the decomposition of the starting material and formation of the unprotected compound 2a′.

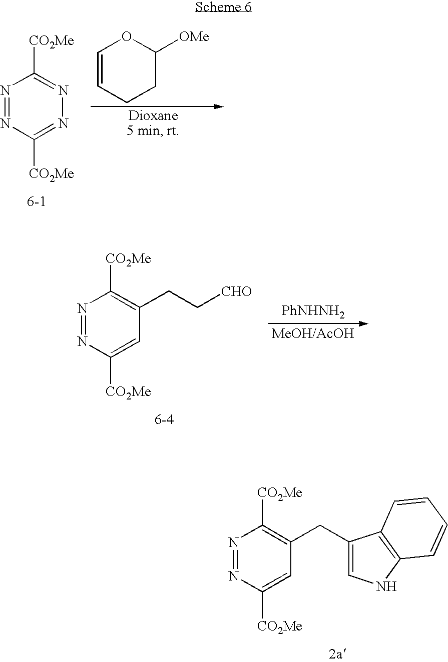

An additional synthetic route employs IEDDAR with a dienophile having an indole ring, or another substituent, already present. Different electron rich dienophiles such as enamines (Geyelin, et al., ARKIVOC 2007, Part 11, 37-45), ketene acetals (Hartmann& Heuschmann, Tetrahedron, 2000, 56:4213-4218), or enol ethers (Akiyama, et al., J. Am. Chem. Soc., 2006, 128:13070-13071) have been employed in this type of [4+2] cycloaddition reaction. Since the conversion of esters into enol ethers by reaction with the Tebbe reagent is a well established procedure (Hartley & McKiernan, J. Chem. Soc., Perkin Trans. 1, 2002, 2763-2793), the N-Boc protected derivative 5 of the commercial available methyl-2-(1H-indol-3-yl)acetate can be selected as the precursor of the electron rich dienophile 6. See Scheme 7 following. Treatment of compound 5 with the Tebbe reagent in tetrahydrofuran at low temperature can afford the desired enol ether 6. Subsequent reaction with substituted tetrazine at room temperature can afford pyridazine 2a. Cmpd 2a′ is obtainable from cmpd 2a by deprotection of the protecting Boc group by methods well known in the art.

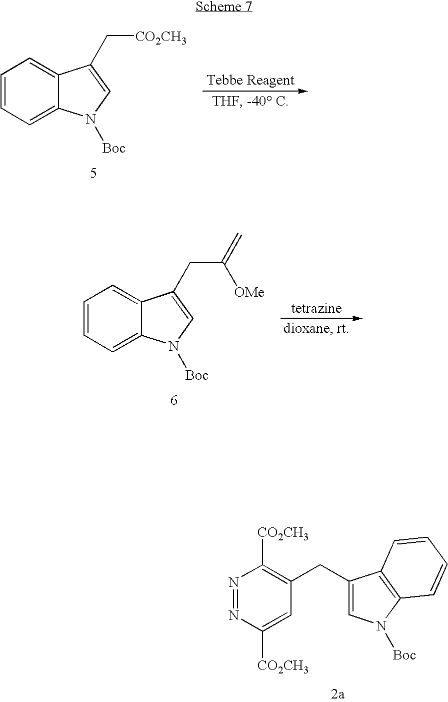

Mono-functionalization of 3,6-dimethylpyridazine dicarboxylates. In the late 1970's, Weinreb and co-workers discovered a method for the general, mild conversion of esters to amides. See e.g., Basha, et al., Tetrahedron Lett., 1977, 48:4171-4174. As shown in Scheme 8 following, this procedure involves the in-situ generation of aluminum amides from an amine and AlMe₃.

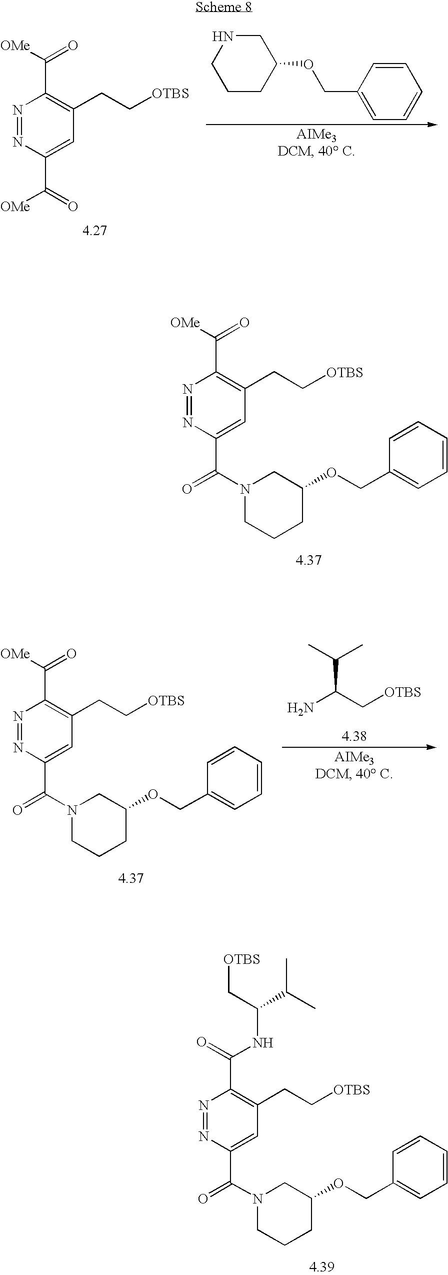

Exposure of the piperidine aluminum amide to a solution of pyridazine diester 4.27 with gentle heating (e.g., 40° C.) results in smooth conversion to the mono-amide 4.37. Modifications have been made to this protocol for use with ammonium hydrochloride salts See e.g., Sidler, et al., J. Org. Chem., 1994, 59:1231-1233; Levin, et al., Synth. Comm., 1982, 12:989-993. Some examples on pyridazine systems have been reported in the literature. See e.g., (Wan, et al., Tetrahedron, 2001, 57:5497-5507).

As further illustrated in the scheme, the reactivity of the remaining C₃-ester towards aluminum amides can be demonstrated by exposing mono-amide 4.37 to TBS-valinol 4.38 in the presence of AIMe₃ to give the diamide-pyridazine 4.39.

As further illustrated in Scheme 9 following, these conditions can also be applied to the 4-isobutyl substituted pyridazine ring 4.21 and piperidine to give the mono-amide 4.121 in good yield. The only other compounds observed by TLC or isolated from this reaction mixture are the diamide and unreacted diester.

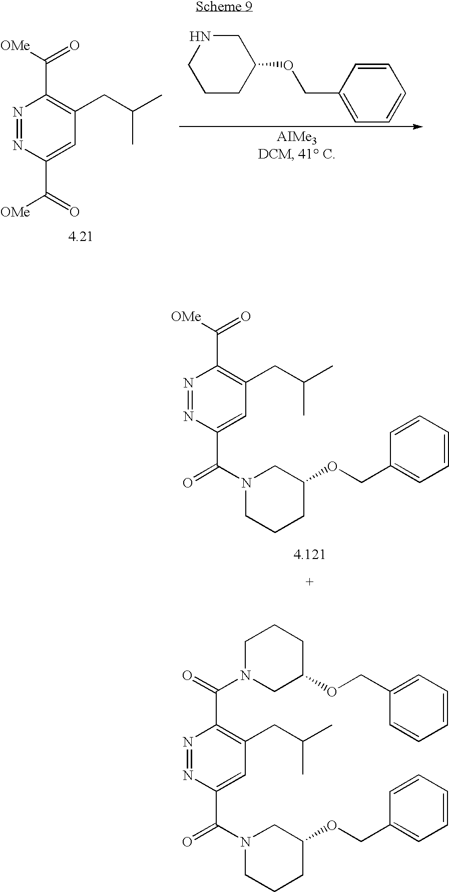

Functionalization of the remaining pyridazine methyl ester. As illustrated in Scheme 10 following, exposure of mono-ester 4.121 to L-valinol in the presence of AIMe₃ can also result in the aminolysis of the methyl ester function, however the yield of this reaction can be quite low. To increase the yield, the methyl ester may be transformed into the acyl hydrazide, diazotized, and displaced with L-valinol to give the di-amide 4.59 with a good overall yield.

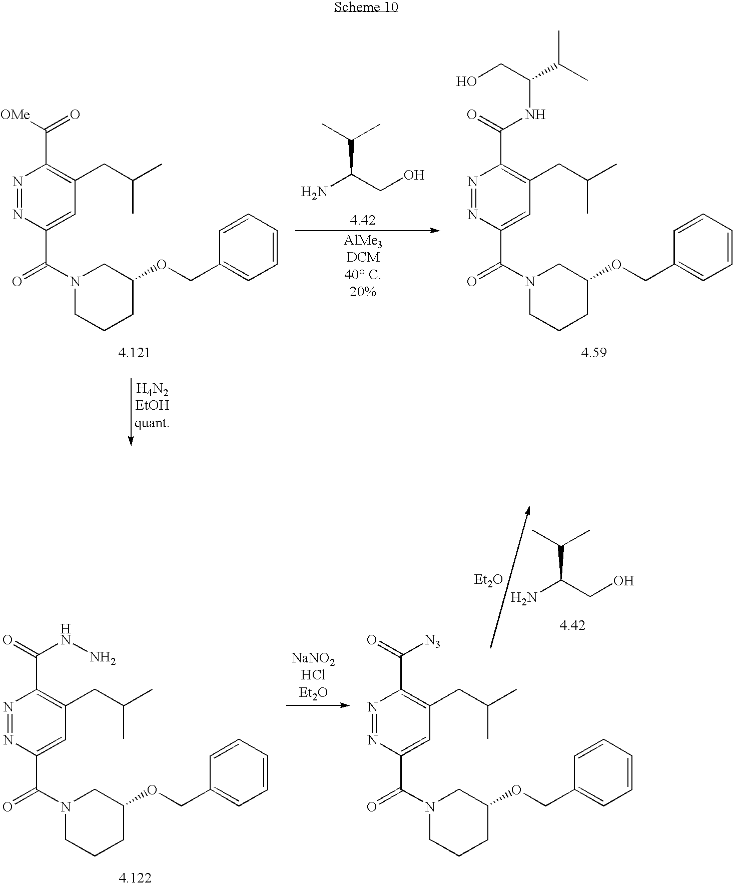

In order to increase the rigidity of the molecule and decrease loss of entropy on binding, the β-hydroxy amide can be closed to form an oxazole. This ring closure is illustrated in Scheme 11 following with the di-amide 4.59. The first step to form an oxazole ring from the β-hydroxy amide moiety requires an oxidation of the alcohol to an aldehyde. This oxidation can be achieved, for example, using the Dess-Martin periodinane. Exposure of the resultant amide-aldehyde to reaction conditions that include PPh₃, 2,6-di-tent-butyl pyridine, dibromo-tetrachloroethane and DBU, can transform the amide-aldehyde into the oxazole 4.73 in good yield.

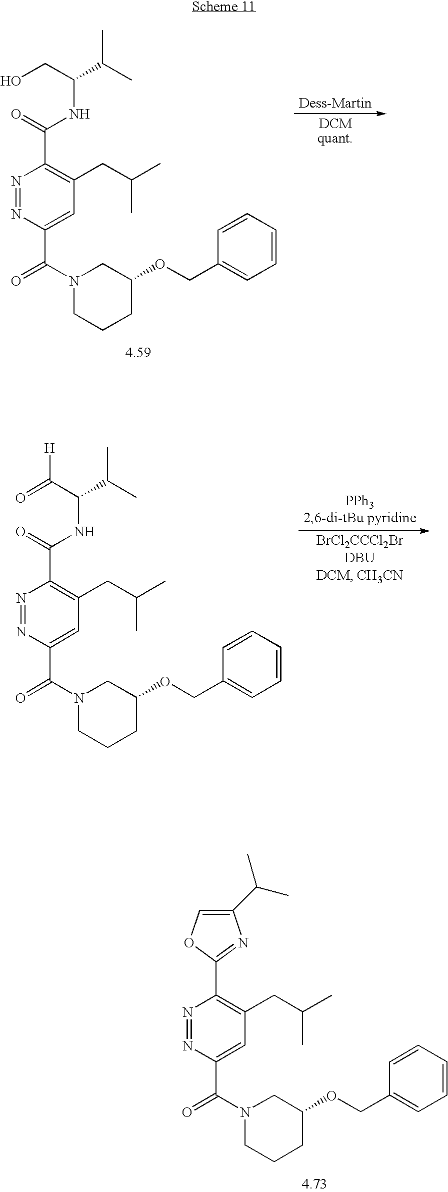

“Symmetrical” difunctionalization of pyridazine dimethyl esters. Provided herein are methods for preparing “symmetrically” substituted pyridazines where the groups at the 3- and 6-positions are equivalent. While AlMe₃ is a very reactive reagent, the yields obtained for aminolysis at the 3-position can be low. However, magnesium chloride is another effective Lewis Acid capable of carrying out the aminolysis of methyl esters. Indeed, exposure of iso-butyl substituted diester 4.21 to an excess of MgCl₂ and L-valinol proceeds smoothly to give the diamide 4.51 in 63% yield. See Example 2. Oxidation to the dialdehyde 4.52 followed by oxazole formation as described above can afford the dioxazole-pyridazine compound 4.53. A generic scheme for difunctionalization of pyridazine dimethyl esters is provided in Scheme 12 following.

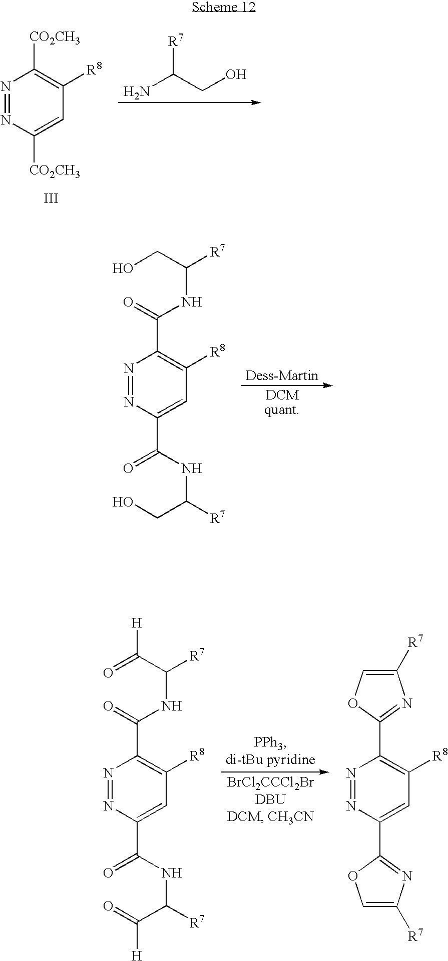

Difunctionalization of pyridazine dimethyl esters. By sequential reaction of the pyridazine with amino alcohol under limiting conditions, with optional isolation of the resulting adducts, difunctionalization of the pyridazine dimethyl ester can be achieved as provided in Scheme 13 following.

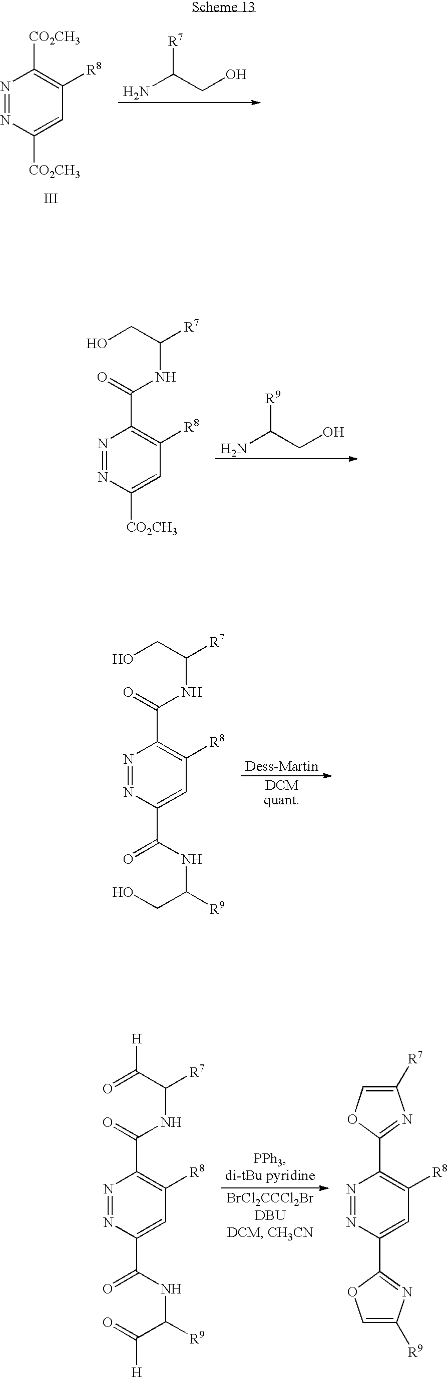

Preparation of a Library of Potential Bak Mimetics, the Synthetic Sequences described herein were applied to combine a series of alpha-helix mimetic scaffolds that are potential mimetics of Bak. Each of the scaffolds has three rings. The central ring is a pyridazine and the outer rings are either piperidines or oxazoles. Each of the rings includes an amino acid side chain or an analogue thereof as a substituent. As known in the art, certain side chains (e.g., Trp-indole, Asn-amide and Tyr-phenol) require protecting groups to be compatible with the synthetic conditions described herein. In some embodiments, the mimetic includes at least one amino acid side chain of a naturally occurring aliphatic amino acid or analog thereof. In some embodiments, the mimetic includes at least one amino acid side chain of a naturally occurring polar amino acid or analog thereof. In some embodiments, the mimetic includes at least one amino acid side chain of a naturally occurring acidic amino acid or analog thereof. In some embodiments, the mimetic includes at least one amino acid side chain of a naturally occurring basic amino acid or analog thereof. In some embodiments, the mimetic includes at least one amino acid side chain of an aromatic amino acid or analog thereof. In some embodiments, the mimetic includes at least one amino acid side chain of a naturally occurring amino acid or analog thereof that contains a hydroxyl or thiol moiety.

### IV. Methods of Use

There are provided herein methods of inhibiting or disrupting the interaction between two proteins. In some embodiments, the method includes inhibiting or disrupting the interaction between an alpha helix of a first protein and the alpha helix binding pocket of a second protein. In some embodiments, the method includes the step of contacting the second protein with a compound as described herein. In some embodiments, the protein-protein interaction involves Bak/Bcl-X_{L}, p53/HDM2, calmodulin/smooth muscle myosin light-chain kinase, or the gp41 assembly.

Thus, in one embodiment, a method of treating a disease, condition or disorder mediated by disrupting a protein-protein interaction is provided. The method includes the step of administering a therapeutically effective amount of a compound provided herein to a patient in need thereof to treat the disease, condition or disorder. In some embodiments, the disease is cancer, a viral infection (e.g. HIV infection), or AIDS. In some embodiments, the cancer is pancreatic, ovarian, liver, skin, bladder, breast, prostate, colorectal and adrenal cancer, B-cell lymphoma, B-cell leukemia, chronic lymphocytic leukemia, multiple myeloma, malignant melanoma, or non-small cell lung carcinoma.

Protein-protein interactions involving an alpha helix of a first protein and an alpha helix pocket of a second protein are well known in the art. Without being limited by any particular theory, the mechanism of binding appears to involve the fitting of the hydrophobic face of a small amphipathic alpha helix of one protein into a well-defined pocket on another protein during their binding to one another. Examples of such interactions include, but are not limited to, protein-protein interactions described herein.

### V. Assays

### Bak/Bcl-XL Assay

The affinity of each molecule within the library of potential Bak mimetics can be assayed with respect to binding to Bcl-x_{L} receptor. The binding affinity can be determined, for example, by a competitive binding assay based on fluorescence polarization. See Wang, et al., Proc. Natl. Acad. Sci., 2000, 97:7124-7129.

Competitive fluorescence polarization-based assays require a dye-labeled peptide to determine if the small molecule has affinity for the target protein. These assays can be carried out with a fluorescein labeled Bid peptide. Bid possesses high sequence homology to Bak, as known in the art. When a solution of fluorescein labeled Bid peptide is irradiated with polarized light, the peptide rotates quickly and the emitted light is depolarized. Upon complexation with the target Bcl-x_{L} protein, the larger complex rotates slowly and the emitted light remains polarized.

When the fluorescein-labeled Bid/Bcl-x_{L} complex is exposed to small molecules with binding affinity for Bcl-x_{L}, the Bid peptide is displaced and the amount of depolarized light emitted increases. This polarization amount (mP) provides an estimate of the affinity for the small molecules for Bcl-x_{L}

Compound HA-1 is a terphenyl alpha-helix mimetic previously shown to bind to Bcl-X_{L} with nanomolar affinity. See Kutzki, et al., J. Am. Chem. Soc., 2002, 124:11838-11839. This compound acts as a positive control.

Of the compounds belonging to the oxazole-pyridazine-piperidine and bis-oxazole scaffold classes, three compounds were particularly active with the desired mP values: 4.73, 4.53 and 4.74. These compounds represent the strongest binders for Bcl-x_{L} of the Bak mimetic library, with structure and mP values shown below.

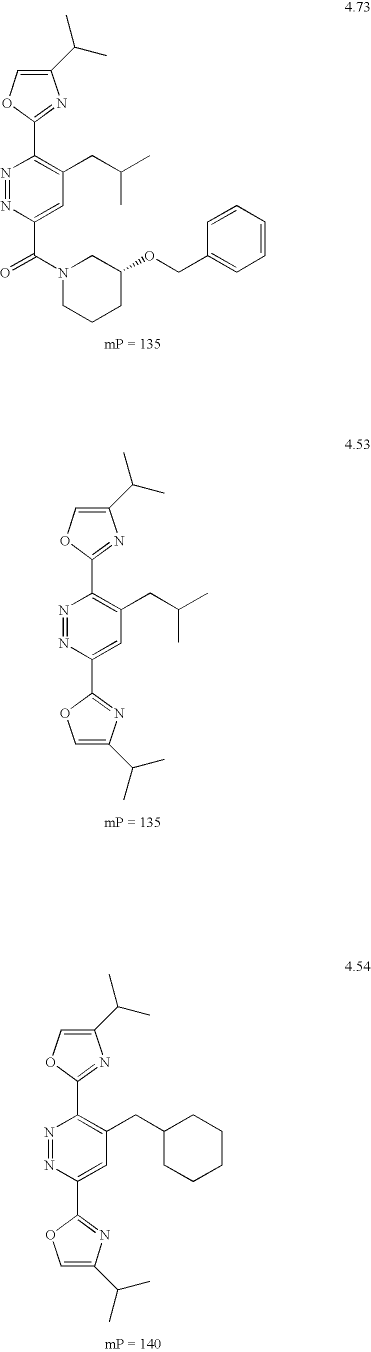

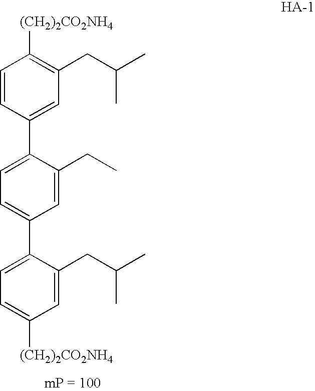

### VI. Pharmaceutical Compositions

Provided herein are pharmaceutical compositions which may be prepared by mixing one or more compounds described herein, pharmaceutically acceptable salts thereof, stereoisomers thereof, tautomers thereof, or solvates thereof, with pharmaceutically acceptable carriers, excipients, binders, diluents or the like to treat or ameliorate a variety of disorders mediated by calcitonin and/or amylin receptors. The compositions of the invention may be used to create formulations and prevent or treat disorders mediated by calcitonin and/or amylin receptors such as bone and metabolic diseases. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by nasal administration, by rectal administration, subcutaneous injection, intravenous injection, intramuscular injections, or intraperitoneal injection. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical Formulations and medicaments may be a spray or aerosol containing an appropriate solvent(s) and optionally other compounds such as, but not limited to, stabilizers, antimicrobial agents, antioxidants, pH modifiers, surfactants, bioavailability modifiers and combinations of these. A propellant for an aerosol Formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical Formulation and/or medicament may also be a powder suitable for reconstitution with an appropriate solution as described above. Examples of these include, but are not limited to, freeze dried, rotary dried or spray dried powders, amorphous powders, granules, precipitates, or particulates. For injection, the Formulations may optionally contain stabilizers, pH modifiers, surfactants, bioavailability modifiers and combinations of these.

For rectal administration, the pharmaceutical Formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of Formulations of the soft gelatin type and suppositories. Water, saline, aqueous dextrose and related sugar solutions, and glycerols may be employed in the preparation of suspension Formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in Remingtons Pharmaceutical Sciences, Mack Pub. Co., New Jersey (1991), which is incorporated herein by reference.

The formulations of the invention may be designed to be short-acting, fast-releasing, long-acting, and sustained-releasing as described below. Thus, the pharmaceutical Formulations may also be Formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical Formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

A therapeutically effective amount of a compound of the present invention may vary depending upon the route of administration and dosage form. The typical compound or compounds of the instant invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

The present invention, thus generally described, will be understood more readily by reference to the following examples, which are provided by way of illustration and are not intended to be limiting of the present invention.

### VII. Examples

General Methods. Commercially available reagent-grade solvents were employed without purification. ¹H and ¹³C NMR spectra were recorded on 300 or 600 MHz spectrometers. Chemical shifts are expressed in ppm (δ), using tetramethylsilane (TMS) as internal standard for ¹H and ¹³C nuclei (δ_{H} and δ_{C}=0.00).

Abbreviations. The following abbreviations are used herein: AcOH: Acetic acid; BuOH: Butanol; cHex: Cyclohexane; DAST: N,N-Diethylaminosulfur trifluoride; DCM: dichloromethane; DIEA: N,N-Diisopropylethylamine; DMF: N,N-Dimethylformamide; DMSO: Dimethylsulfoxide; EDCI: 1-ethyl-3-(3′-dimethylaminopropyl)carbodiimide hydrochloride; EtOAc: ethyl acetate; Et₃N, TEA: Triethylamine; HATU: 2-(7-Aza-1H-benzotriazole-1-yl)-1,1,3,3-tetramethyluronium hexafluorophosphate; Hex: Hexanes; HOBt: 1-Hydroxybenzotriazole; MeOH: Methanol; m.p.: Melting point; NMR: Nuclear magnetic resonance; r.t.: Room temperature; TFA: Trifluoroacetic acid; THF: Tetrahydrofuran; TMS: Trimethylsilyl.

### Example 1

### Synthesis of Substituted Pyridazines

As illustrated in Table 1 following, tetrazine 4.11 was reacted with a variety of alkynes bearing hydrophobic groups under the listed condition to give a series of pyridazines useful in the synthesis of compounds described herein.

TABLE 1
Synthesis of pyridazine compunds.

| Tetrazine | Dienophile | Conditions | Resultant |
|---|---|---|---|
| 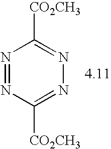 | 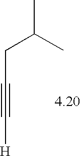 | 80° C. 1,4-dioxane 6 hr 67% | 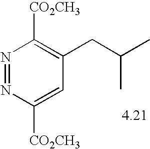 |
| 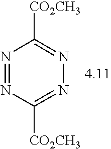 | 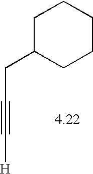 | 80° C. 1,4-dioxane 12 hr 67% | 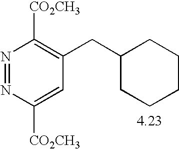 |
| 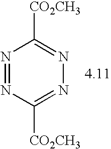 | 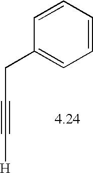 | 80° C. 1,4-dioxane 12 hr 96% | 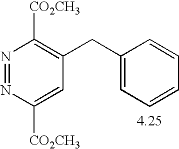 |
| 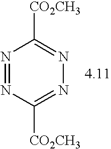 | 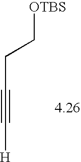 | 80° C. 1,4-dioxane 6 hr 67% | 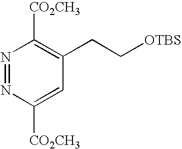 |

### Example 1a

### Dimethyl 4-isobutylpyridazine-3,6-dicarboxylate (4.21)

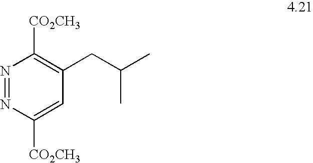

Tetrazine 4.11 (500 mg, 2.52 mmol) was dissolved in 12.5 mL anhydrous 1,4-dioxane. To this bright red solution was added 355 μL 4-methyl pentyne 4.20 (234 mg, 2.85 mmol). The reaction vessel was sealed and heated to 80° C. for 6 hours. The volatiles were removed under reduced pressure and the crude resultant was purified via silica gel chromatography (9.5:0.5 CH₂Cl₂-EtOAc) to give 423 mg (67% yield) of a yellow solid. R_{f}: (9.5-0.5 CH₂Cl₂-EtOAc) 0.33; MS: (MALDI-FTMS) MH⁺ expected: 253.1183, found 253.1187.

### Example 1b

### 3,6-Dimethyl-4-cyclohexyl-1,2-pyridazine dicarboxylate (4.23)

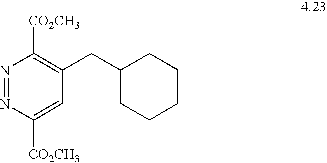

Tetrazine 4.11 (500 mg, 2.52 mmol) and 3-cyclohexyl-1-propyne 4.22 (402 μL, 2.78 mmol, 340 mg) were combined in a sealed tube and dissolved in 12.5 mL 1,4-dioxane. The reaction vessel was sealed and heated to 80° C. for 22 hours. The volatiles were evaporated under reduced pressure and the resultant purified by silica gel chromatography (9:1 DCM-EtOAc, r_{f}=0.5) to give 496 mg of a yellow oil (67% yield). MS: (ESI-TOF) MH⁺ expected: 293.1496, found: 293.1502.

### Example 1c

### 3,6-Dimethyl-4-benzyl-1,2-pyridazine dicarboxylate (4.25)

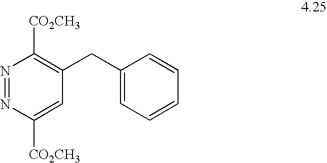

Tetrazine 4.11 (500 mg, 2.52 mmol) and 3-phenyl-1-propyne 4.24 (376 μL, 3.02 mmol, 352 mg) were combined in a sealed tube and dissolved in 12.5 mL 1,4-dioxane. The reaction vessel was sealed and heated to 80° C. for 23 hours. The volatiles were evaporated under reduced pressure and the product purified by silica gel chromatography (9:1 DCM-EtOAc, r_{f}=0.5) to give 690 mg of a dark yellow oil (96% yield). MS: (ESI-TOF) MH⁺ expected: 287.1026, found: 287.1022.

### Example 2

### 2,2′-(4-isobutylpyridazine-3,6-diyl)bis(4-isopropyloxazole) (4.53)

Synthesis of a bis oxazole pyridazine compound is illustrated in Scheme 14 following.

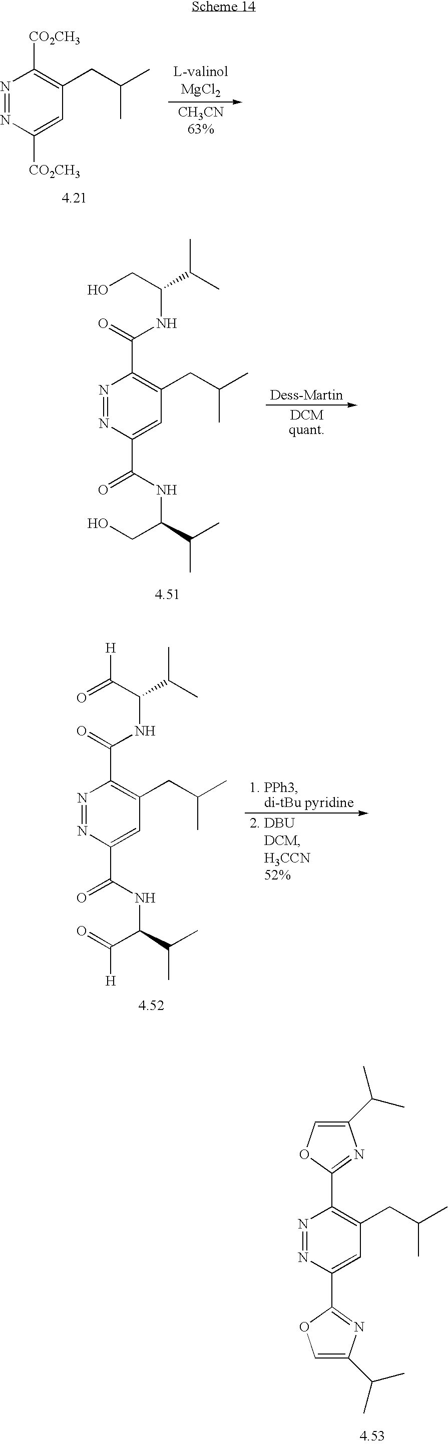

### Step 1.

N3,N6-bis((S)-1-hydroxy-3-methylbutan-2-yl)-4-isobutylpyridazine-3,6-dicarboxamide (4.51). To a stirred solution of compound 4.21 (360 mg, 1.12 mmol) and MgCl₂ (641 mg, 6.75 mmol, 6 eq) in CH₃CN (5 mL) was added dropwise a solution of L-valinol (580 mg, 5.62 mmol) in CH₃CN (5 mL) at room temperature. The mixture was sonicated for 1 hr and then stirred for 24 hr under nitrogen. The mixture was further refluxed for 48 hr. The reaction mixture was poured into water (10 mL) and the aqueous layer was extracted with CH₂Cl₂ (3×10 mL). The organic layers were collected, dried over Na₂SO₄, filtered and concentrated in vacuo. The crude residue was purified on silica (CH₂Cl₂/MeOH 95:5) to afford compound 4.51 as a pale yellow oil (280 mg, 0.71 mmol, 63%); HRMS: (ESI-TOF) MH⁺ expected: 395.2653, found: 395.2644.

### Step 2.

Compound 4.52. To a stirred solution of bis-alcohol 4.51 (150 mg, 0.38 mmol) in CH₂Cl₂ (8 mL) at 0° C. was added Dess-Martin periodinane (484 mg, 1.14 mmol). The mixture was stirred at 0° C. for 30 min, room temperature for 2 hr, washed with aqueous NaHCO₃/Na₂S₂O₃ (1:1, 5 mL), dried (Na₂SO₄), filtered and concentrated to afford the crude di-aldehyde 4.52.

### Step 3.

2,2′-(4-isobutylpyridazine-3,6-diyl)bis(4-isopropyloxazole) (4.53). The di-aldehyde 4.52 was then immediately dissolved in cold CH₂Cl₂ (0° C., 6 mL), and treated with Ph₃P (600 mg, 2.28 mmol) and 2,6-di-tert-butyl pyridine (1.682 mL, 7.61 mmol). Then BrCCl₂CCl₂Br (750 mg, 2.28 mmol) was added. After 1 hr, CH₃CN (7 mL) and then DBU (1.138 mL, 7.61 mmol) were added. The mixture was allowed to warm to room temperature for 2 hr and concentrated. The crude residue was purified by flash chromatography twice (AcOEt and hexane to hexane/AcOEt 8/2) to afford the oxazole as a pale yellow solid (70 mg, 0.19 mmol, 52%). HRMS: (ESI-TOF) MH⁺ expected: 355.2128. found: 355.2127.

### Example 3

### (R)-Methyl 6-(3-(benzyloxy)piperidine-1-carbonyl)-4-isobutylpyridazine-3-carboxylate (4.121)

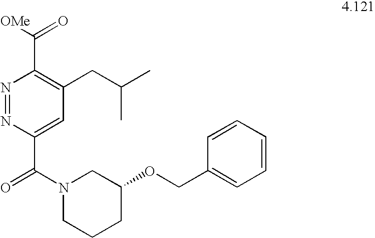

A nitrogen flushed, oven-dried 5 mL schlenk flask was charged with 700 μL anhydrous methylene chloride and 28 μL (R)-3-(benzyloxy)piperidine (0.282 mmol). Slowly, 141 μL Me₃Al (2.0M solution in toluene, 0.282 mmol) was added. The mixture was stirred at room temperature, under nitrogen, for 20 minutes. Dimethyl ester 4.21 (0.282 mmol) was dissolved in 700 μL anhydrous methylene chloride and added as a solution slowly to the aluminum-amide mixture. The schlenk flask was sealed and heated to 41° C. for 24 hours. The reaction was cooled to room temperature, carefully quenched with dilute (1M) HCl, and extracted with methylene chloride. The organic fractions were combined, dried over MgSO₄, and evaporated to dryness under reduced pressure. The crude product was purified via silica gel chromatography with 9.5:0.5 methylene chloride-ethyl acetate eluent to give the desired compound. ¹H NMR: (CDCl₃) δ 7.66 (s, 0.4H), 7.54 (s, 0.6H), 7.36-7.16 (m, 5H), 4.71 (d, J=12 Hz, 0.4H), 4.57 (d, J=12 Hz, 0.4H), 4.43 (d, J=12 Hz, 0.6H), 4.25 (d, J=12 Hz, 0.6H), 7.54 (s, 3H), 3.92-3.85 (m, 0.6H), 3.75-3.70 (m, 1H), 3.65-3.50 (m, 2.4H), 3.40-3.35 (m, 0.4H), 2.82-2.78 (m, 1H), 2.70-2.55 (m, 1H), 2.05-1.55 (m, 4.6H), 0.92 (dd, J=6.6 Hz, 3.0 Hz, 2.5H), 0.83 (dd, J=6.6 Hz, 3.0 Hz, 3.5H); MS: (ESI) MH⁺ expected: 412, found: 412.

### Example 4

### (R)-6-(3-(Benzyloxy)piperidine-1-carbonyl)-4-isobutylpyridazine-3-carbohydrazide (4.122)

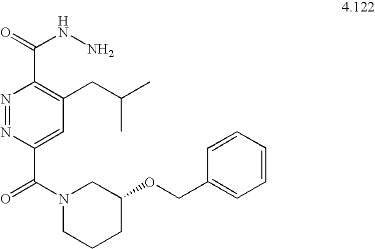

Methyl ester 4.121 (0.106 mmol) was dissolved in 10.5 mL ethanol, followed by the slow addition of 130 μL hydrazine hydrate (65% hydrazine, 2.65 mmol, 85 mg). The reaction mixture was stirred at room temperature under nitrogen for 20 hours, and the volatiles removed under reduced pressure. The crude product was purified using silica gel chromatography (9.5:0.5:0.1% DCM-MeOH-Et₃N) to give the acylhydrazide. ¹H NMR: (600 MHz, CDCl₃) δ 7.67 (s, 0.4H); 7.57 (s, 0.6H) 7.42-7.22 (m, 5H); 4.78 (d, ³J=12 Hz, 0.4H); 4.63 (d, ³J=12 Hz, 0.4H); (d, ³J=12 Hz, 0.6H); 4.31 (d, ³J=12 Hz, 0.6H); 4.28 (m, 0.4H); 3.92-3.91 (m, 0.6H); 3.72-3.37 (m, 5H); 3.10-3.00 (m, 1.4H); 2.82-2.78 (m, 0.5H); 2.10-1.55 (m, 4H); 0.98 (m, 2.5H); 0.83 (m, 3.5H).

### Example 5

### 6-((R)-3-(benzyloxy)piperidine-1-carbonyl)-N—((S)-1-hydroxy-3-methylbutan-2-yl)-4-isobutylpyridazine-3-carboxamide (4.59)

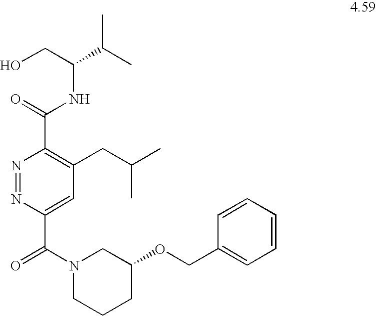

Sodium nitrite (12 mg, 0.164 mmol) was dissolved in 7.5 mL H₂O. The solution was cooled to 0° C., and 150 μL 1.2M HCl (0.180 mmol) was added slowly. The mixture was allowed to stir for 10 minutes, at which time a solution of 50 mg acyl hydrazide 4.122 (0.164 mmol) in 9 mL ethyl ether was added dropwise. The mixture was stirred vigorously for another 10 minutes at 0° C. (the more non-polar acyl-azide can be visualized by TLC, 2:2:0.4 Hex-EtOAc-MeOH). The aqueous layer was removed, and a pre-cooled solution of L-valinol (18.5 mg, 0.180 mmol) in 2 mL ethyl ether was added slowly. The reaction was allowed to warm to room temperature. The reaction mixture was diluted with ethyl ether, and the organic solution was washed with 1M HCl (3×5 mL), saturated NaHCO₃ (3×5 mL), brine (1×5 mL), dried over MgSO₄ and evaporated under reduced pressure to give an oil. The crude product was purified using silica gel chromatography (4:4:0.4 hexanes-ethyl acetate-methanol to give the desired product. ¹H NMR: (600 MHz, CDCl₃) δ 7.66 (s, 0.4H); 7.54 (s, 0.6H) 7.60-7.57 (m, 1H); 7.40-7.20 (m, 5H); 4.78 (d, ³J=12 Hz, 0.4H); 4.64 (d, ³J=12 Hz, 0:4 H); 4.51 (d, ³J=12 Hz, 0.6H); 4.31 (dd, ³J=12 Hz, ⁴J=3 Hz, 0.4H); 4.24 (d, ³J=12 Hz, 0.6H); 3.00-3.35 (m, 8H); 3.20-3.00 (m, 1.5H); 3.70-3.58 (m, 0.5H); 2.20-1.50 (m, 6H); 1.06 (m, 6H); 0.97 (m, 2.5H); 0.88 (m, 3.5H).

### Example 6

### (R)-(3-(Benzyloxy)piperidin-1-yl)(5-isobutyl-6-(4-isopropyloxazol-2-yl)pyridazin-3-yl)methanone (4.73)

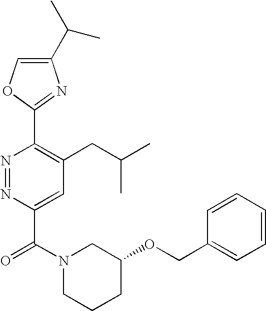

To a stirred solution of β-hydroxyamide 4.59 (0.31 mmol) in CH₂Cl₂ (7 mL) at 0° C. was added Dess-Martin periodinane (195 mg, 0.46 mmol). The mixture was stirred at 0° C. for 30 min, room temperature for 2 hr, washed with aqueous NaHCO₃/Na₂S₂O₃ (1:1, 10 mL), dried (Na₂SO₄), filtered and concentrated to afford the crude aldehyde. The aldehyde was then immediately dissolved in cold CH₂Cl₂ (0° C., 6 mL) and treated with Ph₃P (483 mg, 1.84 mmol) and 2,6-di-tert-butyl pyridine (1.35 mL, 6.13 mmol). Then BrCCl₂CCl₂Br (600 mg, 1.84 mmol) was added. After 1 hr, CH₃CN (7 mL) and then DBU (918 μL, 6.13 mmol) were added. The mixture was then warmed to room temperature for 2 hr and concentrated. The crude residue was purified by flash chromatography twice with (AcOEt to AcOEt/MeOH 9:1) to afford the desired oxazole. ¹H NMR: (600 MHz, CDCl₃) δ 7.70 (s, 0.4H); 7.61 (s, 0.6H) 7.60-7.57 (m, 1H); 7.40-7.20 (m, 5H); 4.74 (d, ³J=12 Hz, 0.4H); 4.61 (d, ³J=12 Hz, 0.4H); 4.45 (d, ³J=12 Hz, 0.6H); 4.37 (dd, ³J=12 Hz, ⁴J=3 Hz, 0.4H); 4.32 (d, ³J=12 Hz, 0.6H); 3.88-3.85 (m, 1H); 3.82-3.75 (m, 1H); 3.70-3.58 (m, 1.6H); 3.50-3.40 (m, 0.8H); 3.14-2.91 (m, 3.4H); 2.13-1.61 (m, 6H); 1.32 (d, ³J=6.6 Hz, 6H); 0.95 (t, ³J=6.6 Hz, 3H); 0.88 (dd, ³J=10 Hz, J=6.6 Hz, 3H).

### Example 7

### Biological Assays

Galanin Assay: Compounds of the present invention can be tested for binding affinity to GalR1 using protocol known in the art, including that described by Land et al. (Methods Neurosci., 1991, 5:225). Many compounds of the invention will demonstrate binding to GalR1. Compounds of the present invention can be tested for ability to displace [¹²⁵I]-galanin from mice hippocampus membranes, which contain all of the galanin receptors, also according to the procedure of Land et al. (Id.). Many such compounds will demonstrate the ability to displace [¹²⁵I]-galanin from mice hippocampus membranes.

Bcl-x_{L}-Bak fluorescence polarization assay. The binding affinity of the molecules for Bcl-x_{L} can be assessed by a fluorescence polarization assay using fluorescein-labeled 16-mer Bak-peptide. See A. M. Petros et al. Protein Science., 2000, 9:2528. Displacement of this probe through competitive binding of the compounds into the hydrophobic cleft of Bcl-x_{L} would lead to a decrease in its fluorescence polarization which in turn can be related to the known affinity of the 16-mer Bak/Bcl-x_{L} complex.

### Example 8

### Formulations

Solution for Parenteral Administration: A solution can be prepared from the following ingredients:

| | |
|---|---|
| Active compound | 5 g |
| Sodium chloride for injection | 6 g |
| Sodium hydroxide | for pH adjustment at pH 5-7 |
| Water for injection. | Up to 1000 mL |

The active constituent and the sodium chloride are dissolved in the water. The pH is adjusted with 2M NaOH to pH 3-9 and the solution is filled into sterile ampoules.

Tablets for Oral Administration: 1000 tablets are prepared from the following ingredients:

| Component | Amount |
|---|---|
| Active compound | 100 g |
| Lactose | 200 g |
| Polyvinyl pyrrolidone | 30 g |
| Microcrystalline cellulose | 30 g |
| Magnesium stearate | 6 g |

The active constituent and lactose are mixed with an aqueous solution of polyvinyl pyrrolidone. The mixture is dried and milled to form granules. The microcrystalline cellulose and then the magnesium stearate are then admixed. The mixture is then compressed in a tablet machine giving 1000 tablets, each containing 100 mg of active constituent.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, any group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While preferred embodiments have been illustrated and described, it should be understood that changes and modifications can be made therein in accordance with ordinary skill in the art without departing from the invention in its broader aspects as defined in the following claims.

What is claimed is:

1. A compound having the structure of Formula (II): 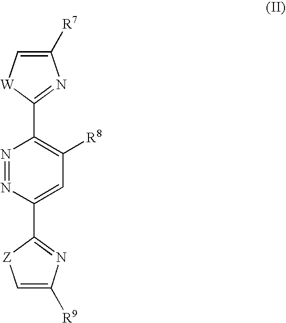
wherein:
W and Z are independently —O— or —S—; and
R⁷, R⁸ and R⁹ are independently hydrogen, substituted or unsubstituted alkyl, substituted or unsubstituted heteroalkyl, or a side chain of a naturally occurring amino acid, homolog thereof, or chemically protected analog thereof.

2. The compound according to claim 1,
wherein:
R⁷, R⁸, and R⁹ are independently hydrogen, R¹⁰-substituted or unsubstituted alkyl, R¹⁰-substituted or unsubstituted heteroalkyl, or a chemically protected analog thereof;
R¹⁰ is independently halogen, —CN, —CF₃, —OH, —NH₂, —SO₂, —COOH, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl;
R¹¹ is independently halogen, —NO₂, —CN, —CF₃, —OH, —NH₂, —SO₂, —COOH, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl; and
R¹² is independently halogen, —NO₂, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl.

3. The compound according to claim 1 having the structure of Formula (IA) 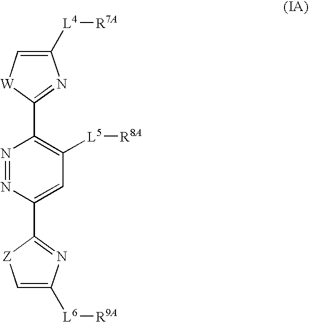
wherein:
L⁴, L⁵ and L⁶ are independently a bond or —O—; and
R^{7A}, R^{8A} and R^{9A} are independently hydrogen, substituted or unsubstituted alkyl, substituted or unsubstituted heteroalkyl, or a side chain of a naturally occurring amino acid, homolog thereof, or chemically protected analog.

4. The compound according to claim 3 having the structure of Formula (IB)

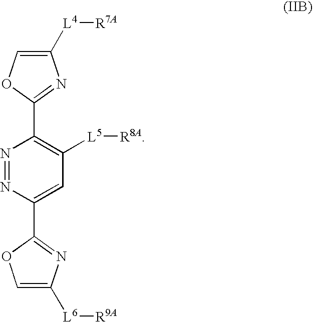

5. The compound according to claim 1,
wherein R⁷, R⁸, and R⁹ are independently —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), —OC(O)—(C₁-C₆ alkyl), or a homolog thereof.

6. A compound according to claim 1 having the structure

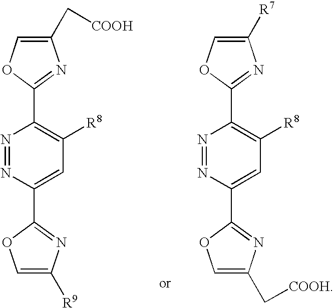

7. A compound according to claim 1 having the structure

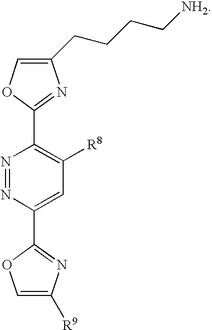

8. The compound according to claim 1 having the structure

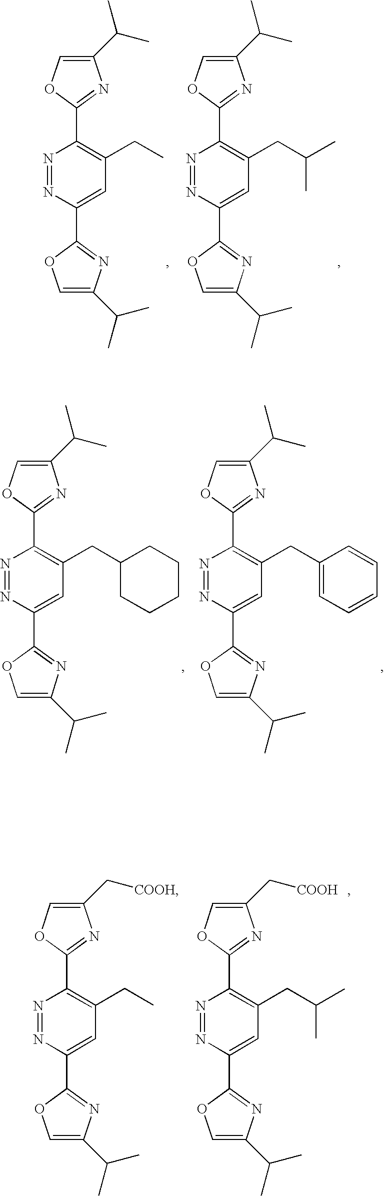

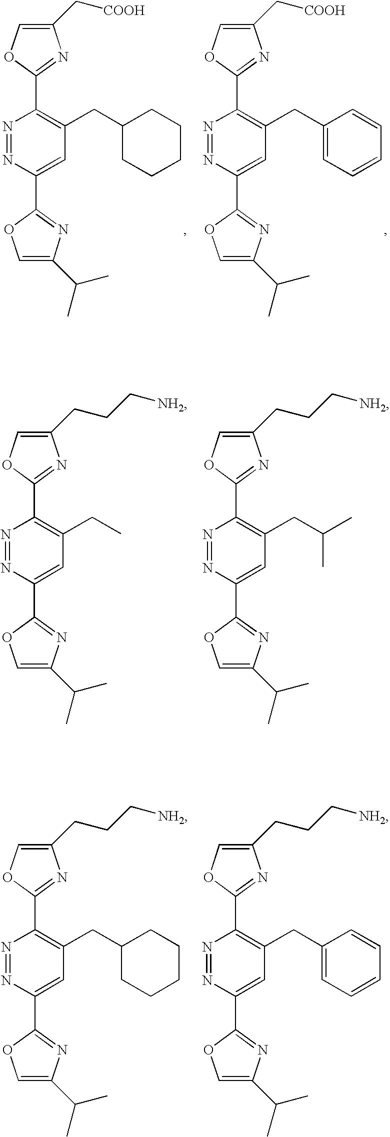

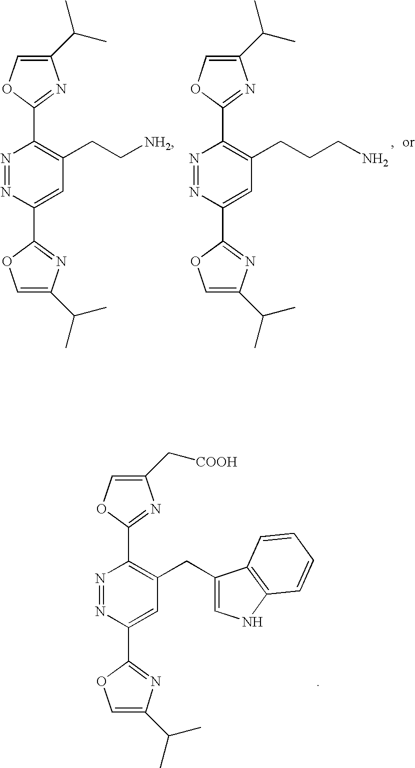

9. A pharmaceutical composition comprising a therapeutically effective amount of a compound according to claim 1 and a pharmaceutically acceptable carrier.

10. A process for synthesizing a compound according to any of claims 1-8 and intermediates thereof, said method comprising reacting a compound of Formula (III) 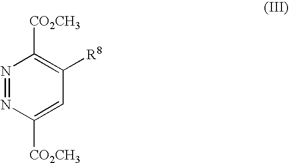
with an amino alcohol, optionally in the presence of a solvent, to form a dicarboxamide, and reacting said dicarboxamide under conditions suitable form an oxazole ring therein.

11. A method for disrupting a protein-protein interaction selected from the group consisting of Bak/Bcl-x_{L}, p53/HDM2, calmodulin/smooth muscle myosin light-chain kinase and gp41 assembly, said method comprising the step of contacting a sufficient amount of a compound having the structure of Formula (II) to disrupt the protein-protein interaction, wherein Formula (II) is 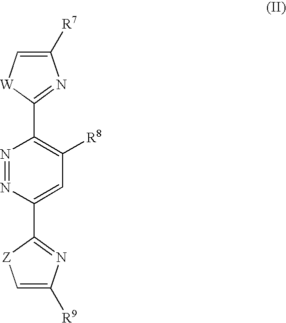
wherein:
W and Z are independently —O— or —S—; and
R⁷, R⁸ and R⁹ are independently hydrogen, substituted or unsubstituted alkyl, substituted or unsubstituted heteroalkyl, or a side chain of a naturally occurring amino acid, homolog thereof, or chemically protected analog.

12. A method for treating conditions and/or disorders mediated by the disruption of the protein-protein interaction according to claim 11, said method comprising the step of administering a sufficient amount to a compound of Formula (II) to a patient to disrupt the protein-protein interaction.

13. The method according to claim 11, wherein said condition is cancer, a viral infection or AIDS.

14. The method according to claim 13, wherein said cancer is pancreatic, ovarian, liver, skin, bladder, breast, prostate, colorectal and adrenal cancer, B-cell lymphoma, B-cell leukemia, chronic lymphocytic leukemia, multiple myeloma, malignant melanoma, or non-small cell lung carcinoma.
